(19) United States
(12) Patent Application Publication
(10) Pub. No.: US 2002/0028455 A1
(43) Pub. Date: Mar. 7, 2002

(54) Methods and reagents for assembling molecules on solid supports

(76) Inventors: Paul E. Laibinis, Medford, MA (US); Ivan H. Lee, Cambridge, MA (US); Leonard S. Lerman, Chestnut Hill, MA (US)

Correspondence Address: FOLEY, HOAG & ELIOT, LLP, PATENT GROUP, ONE POST OFFICE SQUARE, BOSTON, MA, 02109, US

(21) Appl. No.: 09/848,609

(22) Filed: May 3, 2001

Related U.S. Application Data
(60) Provisional application No. 60/201,986, filed on May 3, 2000.

(51) Int. Cl. C12Q 1/68; C07H 21/04; C12M 1/34

(52) U.S. Cl. 435/6; 435/287.2; 536/24.3

(57) ABSTRACT

The present invention makes available methods and reagents for forming support-bound probes, particularly surface bound arrays of molecules, such as nucleic acids, peptides, proteins, small molecules, or other compounds of interest. The method generally involves providing an oligonucleotide bound to the surface of a solid support, hybridizing to the oligonucleotide a complementary sequence covalently linked to a molecule of interest (such as a probe nucleic acid, a peptide, a protein, a small molecule, etc.), and forming a covalent bond between the complementary sequence and the oligonucleotide or the surface of the solid support.

### RELATED APPLICATIONS

This application claims the benefit of the filing date of U.S. Provisional Application No. 60/201,986, filed May 3, 2000.

### BACKGROUND OF THE INVENTION

Many different methods are known for attaching biological molecules to solid supports. See generally, Affinity Techniques, Enzyme Purification: Part B, Meth. Enz. 34 (ed. W. B. Jakoby and M. Wilchek, Acad. Press, N.Y. 1974) and Immobilized Biochemicals and Affinity Chromatography, Adv. Exp. Med. Biol. 42 (ed. R. Dunlap, Plenum Press, N.Y. 1974), incorporated herein by reference. The patent literature also describes a number of different methods for attaching biological molecules to solid supports. For example, U.S. Pat. No. 4,282,287 describes a method for modifying a polymer surface through the successive application of multiple layers of biotin, avidin, and extenders. U.S. Pat. No. 4,562,157 describes a technique for attaching biochemical ligands to surfaces by attachment to a photochemically reactive arylazide. Irradiation of the azide creates a reactive nitrene that reacts irreversibly with macromolecules in solution resulting in the formation of a covalent bond. The high reactivity of the nitrene intermediate, however, results in both low coupling efficiencies and many potentially unwanted products due to nonspecific reactions.

U.S. Pat. No. 4,681,870 describes a method for introducing free amino or carboxyl groups onto a silica matrix; the groups may subsequently be covalently linked to a protein in the presence of a carbodiimide. Also, U.S. Pat. No. 4,762,881 describes a method for attaching a polypeptide chain to a solid substrate by incorporating a light-sensitive unnatural amino acid group into the polypeptide chain and exposing the product to low-energy ultraviolet light.

A variety of techniques have also been developed for attaching oligonucleotides to surfaces. For example, U.S. Pat. No. 4,542,102 describes a method employing a photochemically active reagent (e.g., a psoralen compound) and a coupling agent, which attaches the photoreagent to the substrate. Photoactivation of the photoreagent binds a nucleic acid sequence to the substrate to give a surface-bound probe for a complementary oligonucleotide. However, this method has low quantum yields in protic solvents, lacks spatial directability, and relies upon initial affinity between the photoreagent and nucleic acids to bind the nucleic acids to the surface prior to photoactivation.

U.S. Pat. No. 4,937,188 describes methods for forming maleimide-thiol linkages between a solid support and a molecular tether. In one example, thiol groups on sepharose are reacted with maleimide groups on an RNA polymer, which serves as the tether, and the RNA polymer is reacted with a protein, also via a maleimide-thiol linkage. In another example, maleimide groups immobilized on a solid support are reacted with sulfhydryl RNAse forming RNAse-agarose. U.S. Pat. No. 5,011,770 describes the use of a maleimide-thiol linkage to bind an enzyme label to a binding protein, which in turn binds to single stranded DNA. The patent describes that the binding protein can also be attached to a solid support. The maleimide-thiol linkage is thus spatially separated from the actual linkage between the peptide and solid support.

PCT patent publication No. 90/07582 describes polyacrylamide supports on which are immobilized oligonucleotides. The oligonucleotides are derivatized at the 5′-terminus with thiol groups that are reacted with bromoacetyl groups on the polyacrylamide support during the immobilization process. PCT patent publication No. 91/00868 discloses solid supports on which oligonucleotides are immobilized via a 5′-dithio linkage.

The immobilization of biological polymers on solid supports has also had significant impact on drug discovery and medical diagnostic methods. One important invention in these fields is described in U.S. Pat. No. 5,143,854 and in application Ser. Nos. 624,120, filed Dec. 6, 1990, and 805,727, filed Dec. 6, 1991, and in PCT patent publication No. 90/15070 to Pirrung et al., each of which is incorporated herein by reference. In brief, the invention provides methods and reagents for synthesizing very large numbers of different compounds, particularly biological polymers, in distinct and spatially-addressable locations in a very small area on a substrate. Another description of the invention is found in Fodor et al., Feb. 15, 1991, Science 251:767-773, and the integration of the invention with other drug discovery methods is described in Dower and Fodor, 1991, Ann. Rep. Med. Chem. 26:271-280. A related method uses a photoactivatable derivative of biotin as the agent for immobilizing a biological polymer of interest onto a solid support; see U.S. Pat. No. 5,252,743, and PCT patent publication No. 91/07087 to Barrett et al., each of which is incorporated herein by reference.

Recent approaches to genetic analysis are increasingly placing importance on performing parallel hybridizations in an array format. Applications of the parallel hybridization format include generating diagnostic arrays for tissue typing or diagnosis of genetic disorders (see PCT patent publication No. 89/11548, incorporated herein by reference), DNA sequencing by hybridization, DNA fingerprinting, and genetic mapping (see U.S. patent application Ser. Nos. 624,114, now abandoned and 626,730, filed Dec. 6, 1990, each of which is incorporated herein by reference; see also Khrapko et al., 1991, J. DNA, Seq. Map. 375-388). In these applications of probe arrays, the information content of the array increases as the number of probes is increased. The size limit of the array is dictated by the ability to automate and miniaturize the fabrication of the array.

Many of the above techniques, though, require expensive reagents, or complicated and/or lengthy processing. Thus, there exists a need for improved methods for attaching molecules to a solid support surface, e.g., which do not require complex protocols, complicated protection group schemes, or long preparation times.

### SUMMARY OF THE INVENTION

The present disclosure relates to methods and reagents for generating surface-bound arrays of probes by specifically hybridizing a nucleic acid probe comprising a pairing sequence and a target moiety to a capture oligonucleotide sequence and forming a covalent bond between the surface-bound nucleic acid or the surface and the complementary pairing sequence to covalently attach the probe nucleic acid to the surface.

In certain embodiments wherein the target moiety is a probe nucleic acid, all or part of the probe nucleic acid may be hybridized to a complementary sequence during hybridization to a surface-bound nucleic acid to prevent hybridization of the probe nucleic acid to the surface-bound nucleic acid. After hybridization and covalent crosslinking, the complementary sequence may be dehybridized to expose the single stranded probe sequence covalently attached to the surface.

In certain embodiments, the crosslinking moiety is psoralen.

In certain embodiments, the linking moiety is ethylene glycol.

In certain embodiments, a nucleic acid probe sequence may be continuous with the pairing sequence, e.g., may form a continuous nucleic acid sequence comprising the probe sequence and the pairing sequence. In other embodiments, a linking moiety, such as an alkylene chain, a polyether (e.g., polyethylene glycol), etc., may be used to link the probe sequence to the pairing sequence.

In certain embodiments, the target moiety may be a protein, polypeptide, oligopeptide, small molecule, or any other type of molecule which may bind to a molecule such as a protein, nucleic acid, leptin, antibody, antigen, enzyme, etc.

In certain embodiments, the pairing oligonucleotide comprises between 3 and 50 nucleotides. In certain embodiments, the pairing oligonucleotide comprises between 3 and 20 nucleotides. In certain embodiments, the pairing oligonucleotide comprises between 3 and 10 nucleotides.

In certain embodiments, the solid support bears an array bearing at least 50 probes on the surface thereof In certain embodiments, the solid support bears an array bearing at least 100 probes on the surface thereof. In certain embodiments, the solid support bears an array bearing at least 500 probes on the surface thereof.

In certain embodiments, the solid support bears an array with a probe density of 1-12×10⁻¹¹ moles per cm². In certain embodiments, the solid support bears an array bearing with a probe density of 2-6×10⁻¹¹ probes per cm². In certain embodiments, the solid support bears an array bearing with a probe density of 3-4×10⁻¹¹ probes per cm².

In certain embodiments, the conditions of forming a covalent bond to the surface is exposure to light.

In certain embodiments, the solid support is an array comprises a library of target moieties.

In certain embodiments, the support is functionalized with multiple surface-bound capture oligonucleotides all having the same sequence. In certain embodiments, the support is functionalized with multiple surface-bound capture oligonucleotides with multiple sequences. In certain embodiments, the support is functionalized with multiple surface-bound capture oligonucleotides all having the same sequence.

An aspect of the invention provides for a method of inking a probe to a solid support comprising providing a solid support having an array of surface-bound oligonucleotides, hybridizing to a surface-bound oligonucleotide a probe comprising a pairing oligonucleotide sequence complementary to the surface-bound oligonucleotide sequence and a target moiety, and forming a covalent bond between the pairing oligonucleotide sequence and either the surface-bound oligonucleotide or the solid support.

Another aspect of the invention is a conjugate primer comprising a PCR primer, and an oligonucleotide sequence covalently attached to the PCR primer, and a crosslinking moiety covalently bound to the oligonucleotide sequence and capable of forming a covalent bond to a surface or to an oligonucleotide sequence under predetermined conditions.

Another aspect of the invention provides for a method of forming a self-assembling array of a library of target moieties comprising providing a solid support having an array of surface-bound capture oligonucleotides wherein each capture oligonucleotide having a unique sequence is localized at one or more defined positions on the solid support, contacting the array of surface-bound capture oligonucleotides with a mixture of conjugates comprising a library of target moieties fused to pairing oligonucleotides with sequences complementary to the surface-bound capture oligonucleotides; forming a covalent bond between the pairing oligonucleotide sequence and either the surface-bound capture oligonucleotide or the solid support.

Another aspect of the invention provides a method producing a double stranded DNA sequence with a single stranded overhang comprising contacting a target DNA sequence with a pair of oligonucleotide primers wherein one of the primers is covalently attached to a single stranded oligonucleotide through a linking moiety, and amplifying the target DNA using PCR.

### BRIEF DESCRIPTION OF THE DRAWINGS

FIG. 1 show the relationship between silane composition in solution and silane surface coverage.

FIG. 2 illustrates methods for synthesizing oligonucleotides.

FIG. 3 depicts a PCR primer and PCR probe of the current invention.

FIG. 4 illustrates one embodiment of the invention.

FIG. 5 depicts a method for producing a self-assembling array.

FIG. 6 depicts a stepwise nucleotide reaction efficiency on a surface.

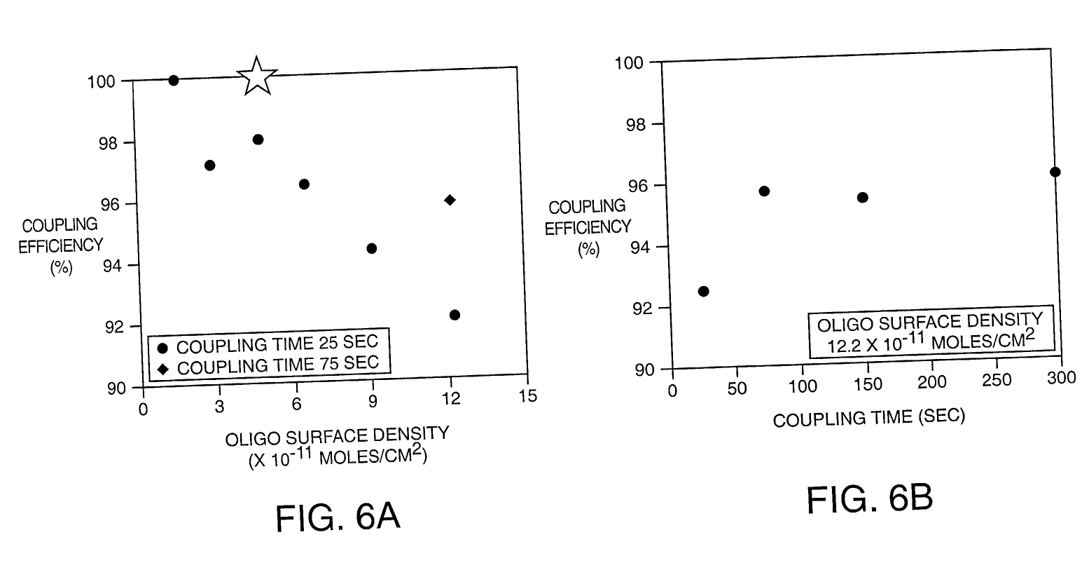

FIG. 7 depicts some oligohybridization conditions.

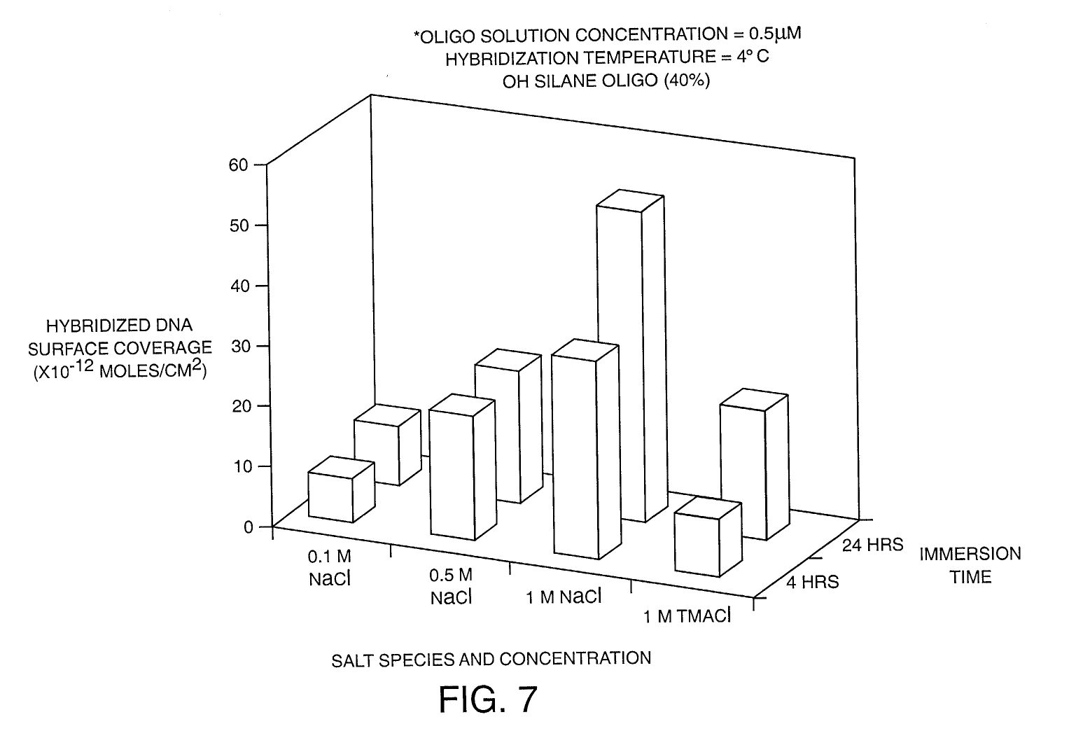

FIG. 8 depicts an aspect of hybridization as a function of chain length.

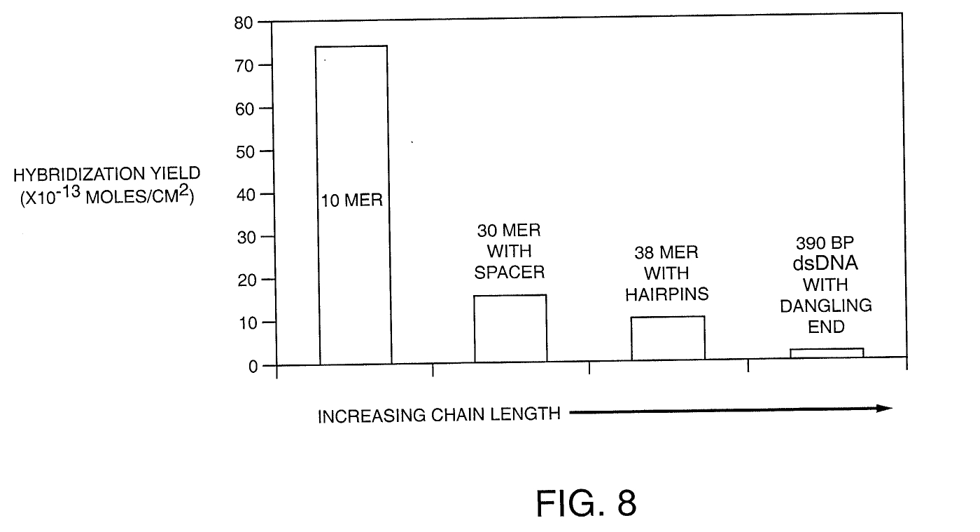

FIG. 9 depicts oligonucleotide coverage as a function of cross-linking.

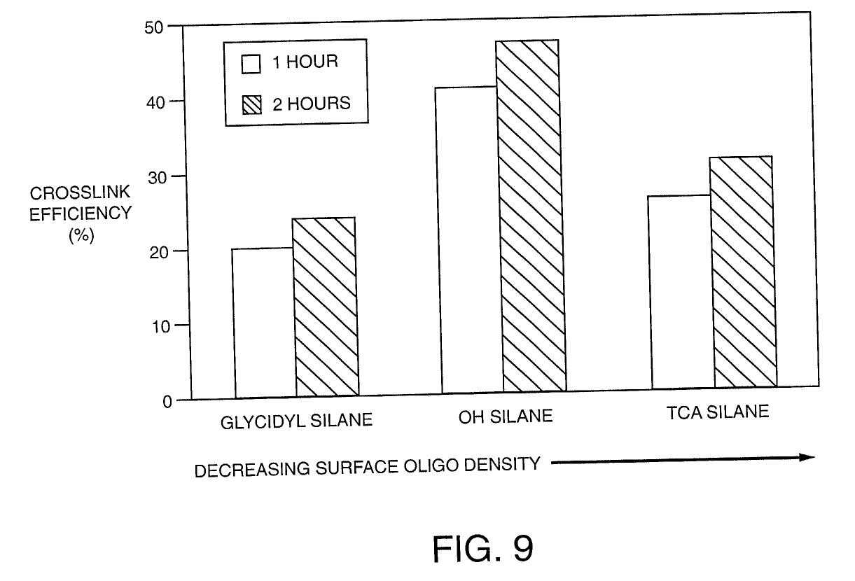

FIG. 10 illustrates the Cy5 fluorescence image of an array.

### DETAILED DESCRIPTION OF THE INVENTION

I. Introduction

The present invention provides a method of covalently attaching molecules of interest to the surface of a solid support without synthesizing the molecules directly on the surface of the support. Instead, molecules of interest can be tethered, directly or through a linking moiety, to a nucleic acid, which may then be hybridized to complementary nucleic acids covalently attached to the surface of a solid support. The hybridized complex may be covalently fixed by crosslinking the hybridized nucleic acids to each other, or by forming a covalent bond between the hybridized nucleic acid and the surface of the solid support. For example, the nucleic acid tethered to the molecule of interest may include a crosslinking moiety, such as psoralen, capable of forming a bond to an oligonucleotide on irradiation with light.

The nature of the specific hybridization interaction makes it possible to direct target molecules to specific regions of a surface which bear oligonucleotides having a predetermined sequence. In this way, an array of target molecules may be synthesized, coupled to oligonucleotides, and disposed in an array on a surface in a predetermined arrangement, e.g., for identifying compounds in a text mixture, analyzing DNA fragments, etc. Because this strategy involves fewer chemical manipulations on the surface of the support, surfaces can be prepared which bear arrays of nucleic acids and peptides having sequences longer than those that can be efficiently, cheaply, and accurately be synthesized directly on the surface of the solid support.

Thus, for preparing arrays of the present invention, it is generally useful to provide a surface bearing an oligonucleotide, or an array of oligonucleotides. Such substrates may be prepared by means well known in the art, or may be purchased. In certain embodiments, the oligonucleotide or oligonucleotides on the surface are conjugated to a crosslinking moiety, such as psoralen, a diazo compound such as a diazoester, or any other moiety capable of forming a covalent bond to an oligonucleotide under preselected conditions, such as addition of a chemical, change of pH, or irradiation with light.

A probe useful for preparing arrays of the present invention includes a target moiety suitable for binding to a substrate. In certain embodiments, the target moiety will be selected to be capable of, or for measuring or determining, binding, e.g., reversible binding, to a predetermined binding partner. Examples of target moieties and binding partners are enzymes and substrates, epitopes and antibodies, carbohydrates and lectins, receptors and ligands, nucleic acids and complementary nucleic acids, etc. Thus, target moieties may be carbohydrates, nucleic acids, proteins, polypeptides, oligopeptides, small molecules which may bind receptors or inhibit enzymes, etc. In preferred embodiments, only one type of target moiety will be selected for any particular surface.

The target moiety is conjugated to a nucleic acid sequence selected to be complementary to a sequence conjugated to a surface of a solid support, also referred to herein as a pairing sequence, in order to form a probe useful for the methods and arrays of the present invention. As described below, any suitable means of conjugating the target moiety to the nucleic acid sequence may be employed. For example, a target moiety and nucleic acid sequence may be directly coupled through a covalent bond, or may be linked by a linking moiety such as an alkyl chain, an oligoether (e.g., oligo, an oligopeptide, or any other suitable linking moiety as described in further detail below. In embodiments wherein the target moiety is a nucleic acid, the target moiety and pairing sequence may form a continuous nucleic acid. In certain such embodiments, the target moiety may comprise RNA, while the pairing sequence may comprise DNA, or vice versa, or both the target moiety and pairing sequence may be of the same type. Additionally, when the target moiety is a nucleic acid, the target moiety may be a double- or single-stranded nucleic acid; after forming a covalent bond to the solid support, the complementary strand may be disengaged from the probe, leaving a single-stranded nucleic acid for hybridization in an assay.

In certain embodiments, the pairing sequence includes a crosslinking moiety capable of forming a bond to a complementary nucleotide sequence, as discussed above, or may include a crosslinking moiety capable of forming a bond to the surface of the solid support. Typically, for forming an array of the present invention, either the pairing sequence or the complementary surface-bound sequence includes a crosslinking moiety, although in certain embodiments, both sequences may include such crosslinking moieties.

Thus, to link a probe to a solid support, a surface-bound sequence is hybridized to a pairing sequence of a probe to form a stable hybrid. Then, a covalent bond is formed between the probe and either the surface or the surface-bound sequence through a crosslinking moiety as discussed above, e.g., by submitting the hybridized pair to predetermined conditions that render the crosslinking moiety reactive, such as by adding a chemical, altering the pH, or exposing the crosslinking moiety to irradiation. At this point, any additional manipulations, such as disengaging a complementary strand from a double-stranded nucleic acid target moiety or deprotection of a reactive moiety, may be performed before using the support-bound probe, e.g., in an assay.

It will be apparent to those skilled in the art that the methods and compositins of the present invention will find application in any of the above-noted processes for solid phase synthesis of biological polymers and other small molecule ligands.

Relevant techniques useful for carrying out the invention are described, e.g., in Sambrook, J., et al. (1989) Molecular Cloning: a Laboratory Manual, 2d Ed., vols 1-3, Cold Spring Harbor Press, N.Y.; Greenstein and Winitz (1961) Chemistry of the Amino Acids, Wiley and Sons, N.Y.; Bodzansky, M. (1988) Peptide Chemistry: a Practical Textbook, Springer-Verlag, N.Y.; Harlow and Lane (1988) Antibodies: A Laboratory Manual, Cold Spring Harbor Press, N.Y.; Glover, D. (ed.) (1987) DNA Cloning: A Practical Approach, vols 1-3, IRL Press, Oxford; Bishop and Rawlings (1987) Nucleic Acid and Protein Sequence Analysis: A Practical Approach, IRL Press, Oxford; Hames and Higgins (1985) Nucleic Acid Hybridisation: A Practical Approach, IRL Press, Oxford; Wu et al. (1989) Recombinant DNA Methodology, Academic Press, San Diego; Goding (1986) Monoclonal Antibodies: Principles and Practice, (2d ed.), Academic Press, San Diego; Finegold and Barron (1986) Bailey and Scott's Diagnostic Microbiology, (7th ed.), Mosby Co., St. Louis; Collins et al. (1989) Microbiological Methods, (6th ed.), Butterworth, London; Chaplin and Kennedy (1986) Carbohydrate Analysis: A Practical Approach, IRL Press, Oxford; Van Dyke (ed.) (1985) Bioluminescence and Chemiluminescence: Instruments and Applications, vol 1, CRC Press, Boca Rotan; and Ausubel et al. (ed.) (1990) Current Protocols in Molecular Biology, Greene Publishing and Wiley-Interscience, N.Y.; each of which is hereby incorporated herein by reference.

II. Definitions

As used herein, the term “alkyl” refers to a saturated hydrocarbon radical which may be straight-chain or branched-chain (for example, ethyl, isopropyl, t-amyl, or 2,5-dimethylhexyl). When “alkyl” or “alkylene” is used to refer to a linking group or a spacer, it is taken to be a group having two available valences for covalent attachment, for example, —CH₂CH₂—, —CH₂CH₂CH₂—, —CH₂CH₂CH(CH₃)CH₂— and —CH₂(CH₂CH₂)₂CH₂—. Preferred alkyl groups as substituents are those containing 1 to 10 carbon atoms, with those containing 1 to 6 carbon atoms being particularly preferred. Preferred alkyl or alkylene groups as linking groups are those containing 1 to 20 carbon atoms, with those containing 3 to 6 carbon atoms being particularly preferred. The term “polyethylene glycol” is used to refer to those molecules which have repeating units of ethylene glycol, for example, hexaethylene glycol (HO—(CH₂CH₂O)₅—CH₂CH₂OH). When the term “polyethylene glycol” is used to refer to linking groups and spacer groups, it would be understood by one of skill in the art that other polyethers or polyols could be used as well (i.e, polypropylene glycol or mixtures of ethylene and propylene glycols).

“Analogs,” in reference to nucleotides, includes synthetic nucleotides having modified base moieties and/or modified sugar moieties, e.g., as described generally by Scheit, Nucleotide Analogs (John Wiley, New York, 1980). Such analogs include synthetic nucleotides designed to enhance binding properties, reduce degeneracy, increase specificity, and the like. In the methods described herein, n designates a fixed position within a single stranded overhang sequence extending from each double stranded nucleic acid segment. Preferably, nucleotide n is selected by digesting a given double stranded nucleic acid segment with a restriction enzyme, e.g., a class IIS restriction endonuclease, to generate a 5′ or a 3′ single stranded overhang sequence corresponding to the cut site, and n is the first or the last unpaired nucleotide in the overhang sequence.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, or other purified preparations of antibodies and recombinant antibodies.

“Antisense” nucleic acids refer to nucleic acids that specifically hybridize (e.g., bind) with cellular mRNA and/or genomic DNA under cellular conditions so as to inhibit expression (e.g., by inhibiting transcription and/or translation). The binding may be by conventional base pair complementarity or, for example, in the case of binding to DNA duplexes, through specific interactions in the major groove of the double helix.

“Biopolymeric compounds” are compounds which are naturally occurring polymeric compounds, or mimetics or analogues of naturally occurring polymeric compounds. Biopolymeric compounds include nucleic acids such as deoxyribonucleic acids, ribonucleic acids, peptide nucleic acids and the like, and peptides, such as oligopeptides, oligopeptides, polypeptides and proteins.

“Capture oligonucleotide” refers to an oligonucleotide bound to a solid support which is designed to specifically hybridize with the pairing oligonucleotide portion of a conjugate.

“Complementary” nucleic acids, as the term is used herein, refers to sequences which have sufficient complementarity to be able to hybridize under highly stringent or mildly stringent conditions, thereby forming a stable duplex.

“Conjugated” shall mean ionically or, preferably, covalently attached.

“Conjugate” as used herein refers to a fusion between a pairing oligonucleotide and target moiety. The conjugate may optionally contain a crosslinking moiety attached to the pairing oligonucleotide and/or a spacer group between the pairing oligonucleotide and the target moiety.

“Conjugate primer” as used herein refers to a specialized nucleic primer which may be used to produce a conjugate wherein the target moiety is a double stranded nucleic acid. In one embodiment, the primer comprises a pairing oligonucleotide fused to a primer useful for PCR amplification of a desired nucleotide sequence. The conjugate primer may optionally contain a crosslinking moiety attached to the pairing oligonucleotide and/or a spacer group between the pairing oligonucleotide and the PCR primer.

A “crosslinking moiety”, as the term is used herein, is a chemical entity that can form a covalent bond to another molecule or to a surface. Usually, crosslinking groups are heterobifunctional and so have different chemical reactivities on either end of the linking group. Crosslinking moieties may be used to construct the conjugate primer. Crosslinking moieties may also be used to covalently attach a conjugate to a capture oligonucleotide via hybridization between the capture oligonucleotide and the pairing oligonucleotide of the conjugate. In various embodiments, the crosslinking agent may be attached to the capture oligonucleotide, the pairing oligonculeotide and/or exogenously added and not covalently attached to either oligonucleotide.

The language “effective amount” of a targeted therapeutic agent or imaging agent refers to that amount necessary or sufficient to eliminate, reduce, or maintain (e.g., prevent the spread of) an infection, tumor, or other target. The effective amount can vary depending on such factors as the disease or condition being treated, the particular targeted constructs being administered, the size of the subject, or the severity of the disease or condition. One of ordinary skill in the art can empirically determine the effective amount of a particular compound without necessitating undue experimentation.

“Human monoclonal antibodies” or “humanized” murine antibodies, as the terms are used herein, refer to murine monoclonal antibodies “humanized” by genetically recombining the nucleotide sequence encoding the murine Fv region (i.e., containing the antigen binding site) or the complementarity-determining regions thereof with the nucleotide sequence encoding at least a human constant domain region and an Fc region, e.g., in a manner similar to that disclosed in European Patent Application Publication No. 0,411,893 A3. Some additional murine residues may also be retained within the human variable region framework domains to ensure proper target site binding characteristics. Humanized antibodies are recognized to decrease the immunoreactivity of the antibody or polypeptide in the host recipient, permitting an increase in the half-life and a reduction in the possibility of adverse immune reactions.

A “modified nucleotide,” as used herein, refers to a nucleotide that has been chemically modified, e.g., a methylated nucleotide.

“Nucleic acid” refers to polynucleotides, such as deoxyribonucleic acid (DNA) and, where appropriate, ribonucleic acid (RNA). The term should also be understood to include, as equivalents, analogs of either RNA or DNA made from nucleotide analogs, and, as applicable, to the embodiment being described, single- (sense or antisense) and double-stranded polynucleotides. The term encompasses oligonucleotides, e.g., sequences comprised by less than or equal to about 100 bases.

“Nucleotide” is an art-recognized term and includes molecules which are the basic structural units of nucleic acids, e.g., RNA or DNA, and which are composed of a purine or pyrimidine base, a ribose or a deoxyribose sugar, and a phosphate group.

The term “oligonucleotide” as used herein includes linear oligomers of natural nucleotides or analogs thereof, as well as universal nucleotides, including deoxyribonucleosides, ribonucleosides, anomeric forms thereof, peptide nucleic acids (PNAs), and the like, capable of specifically binding to a target polynucleotide by way of a regular pattern of monomer-to-monomer interactions, such as Watson-Crick type base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g., 3-4, to several tens of monomeric units. Analogs of phosphodiester linkages include phosphorothioate, phosphorodithioate, phosphoranilidate, phosphoramidate, and the like.

“Pairing oligonucleotide” refers to a single stranded nucleotide sequence which is covalent attached to a PCR primer, or a target moiety, and is designed to specifically hybridize with a capture oligonucleotide bound to a solid support.

A “peptide” is a polymer in which the monomers are amino acids and are joined together through amide bonds, alternatively referred to as a polypeptide. When the amino acids are α-amino acids, either the L-optical isomer or the D-optical isomer may be used. Additionally, unnatural amino acids, for example, β-alanine, phenylglycine and homoarginine are also meant to be included. Peptides are two or more amino acid monomers long and are often more than 20 amino acid monomers long. Standard abbreviations for amino acids are used (e.g., P for proline). These abbreviations are included in Stryer, Biochemistry, Third Ed., 1988, which is incorporated herein by reference for all purposes.

A “predefined region”, as the term is used herein, is a localized area on a surface which is or is intended to be activated. The predefined region may have any convenient shape, e.g., circular, rectangular, elliptical, etc.

The term “protecting group” as used herein, refers to any of the groups which are designed to block one reactive site in a molecule while a chemical reaction is carried out at another reactive site. More particularly, the protecting groups used herein can be any of those groups described in Greene, et al, Protective Groups In Organic Chemistry, 2nd Ed., John Wiley & Sons, New York, N.Y., 1991, incorporated herein by reference. The proper selection of protecting groups for a particular synthesis will be governed by the overall methods employed in the synthesis. For example, in “light-directed” synthesis, discussed below, the protecting groups will be photolabile protecting groups such as dimethoxybenzoin, NVOC, MeNPOC, and those disclosed in Application PCT/US93/10162 (filed Oct. 22, 1993), incorporated herein by reference. In other methods, protecting groups may be removed by chemical methods and include groups such as FMOC, DMT and others known to those of skill in the art.

A “receptor” is a molecule that has an affinity for a ligand. Receptors may be naturally-occurring or manmade molecules. They can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants, viruses, cells, drugs, polynucleotides, nucleic acids, peptides, cofactors, lectins, sugars, polysaccharides, cellular membranes, and organelles. Receptors are sometimes referred to in the art as anti-ligands. As the term “receptors” is used herein, no difference in meaning is intended. A “ligand-receptor pair” is formed when two molecules have combined through molecular recognition to form a complex.

“Small molecule” refers to a compound which has a molecular weight of less than about 2000 amu, preferably less than about 1000 amu, and even more preferably less than about 500 amu.

A “surface”, as the term is used herein, is any generally two-dimensional structure, e.g., curved, flat, etc., on a solid substrate and may have steps, ridges, kinks, terraces, and the like without ceasing to be a surface.

A “target moiety” is a solvated molecule or particle that is recognized by a particular molecule. Examples of target moieties that can be investigated with the present invention include, but are not restricted to, agonists and antagonists for cell membrane receptors, toxins and venoms, viral epitopes, hormones, opiates, steroids, hormone receptors, peptides, enzymes, enzyme substrates, cofactors, drugs, lectins, sugars, oligonucleotides, oligosaccharides, proteins, and monoclonal antibodies. Target moieties include particles with surfaces that expose the complementar nucleic acid base sequence required for it to become attached.

III. Solid Support Surfaces

The solid substrate may be biological, nonbiological, organic, inorganic, polymeric, or a combination of any of these, existing as particles, strands, precipitates, gels, sheets, tubing, spheres, containers, capillaries, pads, slices, films, plates, slides, etc. The solid substrate is preferably flat but may take on alternative surface configurations. For example, the solid substrate may contain raised or depressed regions on which synthesis takes place. In some embodiments, the solid substrate will be chosen to provide appropriate light-absorbing characteristics. For example, the substrate may be a polymerized Langmuir Blodgett film, functionalized glass, Si, Ge, GaAs, GaP, SiO₂, SiN₄, modified silicon, or any one of a variety of gels or polymers such as (poly)tetrafluoroethylene, (poly)vinylidendifluoride, polystyrene, polycarbonate, or combinations thereof. Other suitable solid substrate materials will be readily apparent to those of skill in the art. Preferably, the surface of the solid substrate will contain reactive groups, which could be carboxyl, amino, hydroxyl, thiol, or the like. More preferably, the surface will be optically transparent and will have surface Si—OH functionalities, such as are found on silica surfaces. In one embodiment, the solid substrate is porous.

The derivatization reagent can be attached to the solid substrate via carbon-carbon bonds using, for example, substrates having (poly)trifluorochloroethylene surfaces, or more preferably, by siloxane bonds (using, for example, glass or silicon oxide as the solid substrate). Siloxane bonds with the surface of the substrate are formed in one embodiment via reactions of derivatization reagents bearing trichlorosilyl or trialkoxysilyl groups.

The particular derivatization reagent used can be selected based upon its hydrophilic/hydrophobic properties to improve characteristics of the invention. As noted above, the derivatization reagent, prior to attachment to the solid substrate, has a substrate attaching group at one end, and a reactive site at the other end. The reactive site will be a group which is appropriate for attachment to a oligonucleotide. For example, groups appropriate for attachment to a silica surface would include trichlorosilyl and trialkoxysilyl functional groups. Groups which are suitable for attachment to a linking molecule include amine, hydroxyl, thiol, carboxylic acid, ester, amide, epoxide, isocyanate and isothiocyanate. Additionally, for use in synthesis, the derivatization reagents used herein will typically have a protecting group attached to the reactive site on the distal or terminal end of the derivatization reagent (opposite the solid substrate). Preferred derivatization reagents include aminoalkyltrialkoxysilanes, aminoalkyltrichlorosilanes, hydroxyalkyltrialkoxysilanes, hydroxyalkyltrichlorosilanes, carboxyalkyltrialkoxysilanes, polyethyleneglycols, epoxyalkyltrialkoxysilanes, and combinations thereof. Any of the above functional groups which forms a bond to the surface may be used as a crosslinking moiety on a capture oligonucleotide or conjugate moiety to form a covalent bond to the surface, as discussed in greater detail below. In one embodiment, the derivatization reagent may include a chemical group, e.g. acetate or trichloroacetate) that can be used to reveal a functional group, after attachment of the derivatizing agent.

In certain embodiments of the invention, polymer-coated supports may be employed. The polymers used for coating a solid support include, but are not limited to polyurethanes, polyesters, polycarbonates, polyureas, polyamides, polyethyleneimines, polyarylene sulfides, polysiloxanes, polyacrylamides, polyimides, polyacetates, or other polymers which will be apparent upon review of this disclosure. The polymers used to coat a solid support are typically repeats of a single monomers which is crosslinked with a second molecule to provide structural integrity to the polymer.

The present invention also provides a method of preparing surfaces in which a polymer, having synthesis properties similar to a commercial peptide resin is attached to a solid support. The polymer films provide a porous three-dimensional matrix functionalized with reactive groups that serve as starting points for oligonucleotide synthesis. The use of an organic polymer on a solid surface will provide greater solvent compatibility and flexibility of the reaction site for attachment of the synthesis building blocks. Another advantage is the potential improvement in surfaces for performing bioassays which results from the variety of polymers available and the degrees of polymer porosity which can be obtained. The extent of binding of target molecules (receptors) to the immobilized oligonucleotide or peptide sequences (ligands) may be substantially increased, which enhances detection, and the multiplicity of binding sites within the polymer support may provide additional kinetic enhancement. Thus surfaces can be designed and prepared for optimum properties in a particular assay. This optimization will take advantage of the relatively thick but loosely woven polymer network that allows macromolecules to diffuse in and out of a layer of tethered ligands. Still other potential advantages that may be achieved with polymer-coated surfaces prepared by the present methods involve improved processing for reusing the surface, easier characterization of the surface for quality control in synthesis, and reduction of potential problems associated with the use of glass surfaces.

A variety of approaches can be employed for the preparation of polymer-coated solid surfaces. In one approach, the solid surface used is a rigid polymer which is then crosslinked with a “soft” polymer layer to confer desired surface properties. Alternatively, a solid surface such as a glass slide can be coated with a polymer film to form a composite. These composites can be created by covalently crosslinking the polymer to silanized glass, by in situ polymerization of monomers on a silanized glass surface, or by relying on the mechanical strength of a polymer film to completely wrap and adhere to a slide that has been dipped.

In any of these methods, the choice of available surface polymers is extensive. Suitable polymers include chloromethylated styrene-divinylbenzene (Merrifield resin), phenylacetamidomethylated styrene-divinylbenzene (PAM resin), and crosslinked polyethylene glycol-polystyrene grafts (TentaGel resin). Other polymers can be prepared as described below.

For purposes of the instant invention, the polymer must carry functional groups which are appropriate for the initiation of peptide, oligonucleotide or other small molecule synthesis. As a result, the polymer must be stable in the presence of the solvents and activating reagents used. Additionally, the surface should exhibit low non-specific binding of receptors.

The polymers which are used to coat the solid support can also be selected based upon their functional groups which will serve as synthesis initiation sites. Typically, polymers having primary amine, carboxyl or hydroxyl functional groups will be selected.

Polymers having hydroxyl functional groups are useful and the resulting surfaces are extremely wettable. Examples of suitable polymers include polyethyleneglycol (PEG, see Rapp Polymere Catalogue, Harris, J., J. Polym. Sci. Polym. Chem. Ed. 22:341 (1984); and Pillai, V., et al., J. Org. Chem. 45:5364-5370 (1980)); poly(vinyl alcohol); and carbohydrates (see J. Chemical Society Chem. Comm., p. 1526 (1990)).

The preparation of thin polymer films on solid surfaces can be accomplished using a variety of methods including dip coating, covalent attachment and in situ polymerization.

Films can be created on solid substrates by dip coating with the polymer solution, followed by evaporation of the solvent and stabilization of the coating using crosslinking agents or UV treatment. Suitable crosslinking agents will depend on the nature of the functional groups present in the polymer. The degree of polymer crosslinking can be varied to produce films which are optically transparent and of uniform thickness. A related method of producing a uniform thickness polymer coating utilizes a spin-coating technique. Another method for the preparation of polymer films on solid surfaces involves preparation of the selected polymer followed by covalent attachment of the polymer to functional groups which are present on the substrate surface or modified substrate surface. Glass surfaces which have been modified with aminopropylsilanes can be reacted with polymers having carboxylic acid functional groups (using, for example, water soluble carbodiimides) or by direct reaction of the modified surface with a polymer having attached anhydride groups. In other embodiments, glass surfaces can be silanized with silanes having epoxide functional groups and subsequently reacted directly with polymers having either amine or hydroxy functional groups. In addition, glass surfaces can be planarized by using polysiloxanes known as “spin on glasses” which may provide both a more uniform planar surface and/or a substantially higher density of functional sites which further provides for better subsequent derivatization. For polymers that are not commercially available, polymer-coated substrates can be formed by carrying out the polymerization reaction on the substrate surface. For example, a mixture of appropriate monomers are dissolved in solvent with an initiator. After a suitable activation period, the solid substrate is dipped into the mixture and then cured at elevated temperatures to complete the polymerization. The resulting surfaces are then washed, dried and functionalized to provide functional groups which are useful as synthesis initiation sites.

Still other methods of preparing polymer-coated solid supports will use combinations of two or more of the above methods. For example, a polymer film can be “grafted” onto a glass support by first silanizing the glass with an acrylamido-alkyl trialkoxysilane. Subsequent polymerization of an acrylamide copolymer layer on top of the silanized glass provides a covalently attached film which exhibits excellent resistance to all of the conditions used for oligonucleotide synthesis, deprotection and hybridization.

The polymer-coated support can be tailored to provide optimal properties for synthesis and for biological assays. For example, the final concentration of functional groups (e.g. hydroxyl) in the polymer can be controlled by varying the relative amounts of nonfunctionalized and functionalized monomers used in forming the polymer. Additionally, the porosity and solubility of the polymer films can be controlled by varying the concentrations of monomers and crosslinking agents in the composition. Thus, a high degree of crosslinking gives a rigid insoluble polymer with low pore size, whereas omitting the crosslinking agent altogether will result in soluble linear polymer chains (with functional groups) extending off the surface of the substrate from the attachment sites.

One of skill in the art will understand that the present invention is not limited to planar surfaces, but is equally useful when applied to other surfaces, for example, spherical beads.

In still another aspect, the present invention provides solid surfaces which are coated with a layer of high molecular weight (500 Kd) dextran (α1-6 poly D-glucose). Solid surfaces which are coated with dextran or other glycans provide more hydrophilic surfaces which exhibit improved characteristics for monitoring the binding of a receptor to a support-bound ligand.

In general, the glycan-coated surfaces can be prepared in a manner analogous to the preparation of polymer-coated surfaces using covalent attachment. Thus, a glass surface can be modified (silanized) with reagents such as aminopropyltriethoxysilane to provide a glass surface having attached functional groups (in this case, aminopropyl groups). The modified surface is then treated with a solution of a modified dextran to provide a surface having a layer of dextran which is covalently attached.

The method of covalently attaching a dextran or other carbohydrate to the glass surface can be carried out using a variety of chemical manipulations which are well known to those of skill in the art. In one embodiment, the surface is modified to produce a glass surface having attached primary amine groups using reagents such as aminopropyltriethoxysilane. The resulting amines are then reacted (using water soluble carbodiimides) with dextrans which have been previously modified with carboxymethyl groups. In another embodiment, the glass surface is modified with hydroxy groups using reagents such as hydroxypropyltriethoxysilane. Subsequent reaction of the hydroxy moiety with epichlorohydrin provides a surface having attached epoxide functional groups. The epoxides can then be reacted directly with hydroxyl groups present in dextran to provide covalent attachment of the dextran to the modified surface.

Following covalent attachment of the dextran to the glass surface, the carbohydrate can be further derivatized to provide synthesis initiation sites for peptide, oligonucleotide or other small molecule synthesis. For example, treatment of dextran-modified surfaces with bromoacetic acid results in derivatives having attached carboxymethyl groups. The carboxylic acid groups can be used as synthesis initiation sites or they can be further modified with lower diaminoalkanes to provide primary amines as synthesis initiation sites. See, Cass, et al., In Peptides: Chemistry, Structure and Biology, Hodges, et al., eds., ESCOM, Leiden pp. 975-977 (1994).

In one embodiment, reactive surface films or self assembled monolayers (SAM) controllable surface density are used on the solid support surface.

In a preferred embodiment, polished silicon chips with <100> orientation are used for the solid substrate. A native oxide layer about 50 Å in thickness provides the covalent attachment sites for the organosilanes, while the underlying reflective silicon surface can be used for ellipsometry measurements. In addition, the relatively flat silicon surface provides a more accurate determination of surface energy using contact angle measurements, because the liquid contact angles are affected by both surface roughness and heterogeneity. Also, the silicon chips are easy to cut and shape compared to glass slides, and chemical dopants in the silicon chips make them semiconductors and thus more readily characterized by XPS. Fluorescence imaging tests conducted on silicon surfaces with silane adlayers showed very low levels of background noise, demonstrating the potential of the surfaces for epifluorescence spectroscopy.

A variety of organosilane reagents can be used to produce SAMs with varying hydroxyl content, where the hydroxyl groups are the active sites for the attachment of activated phosphoramidites in the stepwise oligo synthesis reaction. In one embodiment both GOPS (3-glycidoxypropyl)triethoxysilane) and hydroxyl-terminated alkoxysilanes are used.

In a preferred embodiment, surfaces were prepared by expressing a controlled mixture of hydroxyl and methyl groups by immersing the silicon surfaces in mixtures of two trichlorosilanes containing methyl and TCA (trichloroacetyl) protected hydroxyl end groups. The TCA groups were removed with a mild base to expose the hydroxyl groups. The silane adlayers were characterized by ellipsometry to measure their film thickness, and by wetting measurements with water to measure their hydrophilicity, shown in Table 1.

TABLE 1

| | | Glycidyl silane | | | TCA silane mixtures | |
|---|---|---|---|---|---|---|
| | | Before | After | | Before | After |
| Species | Bare | ring | ring | OH | depro- | depro- |
| Status | SiO2 | opening | opening | silane | tection | tection |
| Thickness | 1 ± 1 | 12 ± 2 | 12 ± 2 | 15 ± 3 | 20 ± 3 | 20 ± 3 |
| (Anstroms) | | | | | | |
| θₐ in water | <10 | 55 | 33 | 50 | 98 | 71-98 |
| (degrees) | | | | | | |
| θ_{rd} in | — | — | — | — | 81 | 48-55 |
| water | | | | | | |
| (degrees) | | | | | | |

In one embodiment, silicon surfaces were exposed to solution mixtures of dodecyl trichlorosilane and trichloroacetyl (TFA) or trifluoroacetyl (TCA) undecyl trichlorosilane (TCS) to produce SAM with mixed surface compositions. Contrary to the monolayer coverage obtained using alkanethiol SAMs, the thickness of mixed TCS SAMs varied with immersion times. For immersions times lower than 1 hour the SAM thickness was approximately 25-30 Å, corresponding to a monolayer level surface coverage. However, as immersion times increased the thickness increased, until at 8 hour immersion the adlayer thickness was above 100 Å, in the multiplayer film regime. TCS may be much more reactive then the triethoxy or trimethoxy silanes and thus more extensive crosslinking between silane chains occur, resulting in gel-like structures over longer reaction times. Therefore the reaction times for TCS were limited to 30 to 60 minutes to prevent multilayer formation. In general the kinetics of TFA TCS SAM formation were more rapid compared to the TCA TCS SAMs, possibly due to the lower chemical stability of the TFA protecting group, which is more readily removed by trace amounts of residual water to prematurely expose the hydroxy group, resulting in unwanted crosslinking side reactions. In one embodiment, the appropriate functional group, e.g. hydroxy, is not generated in the first monolayer.

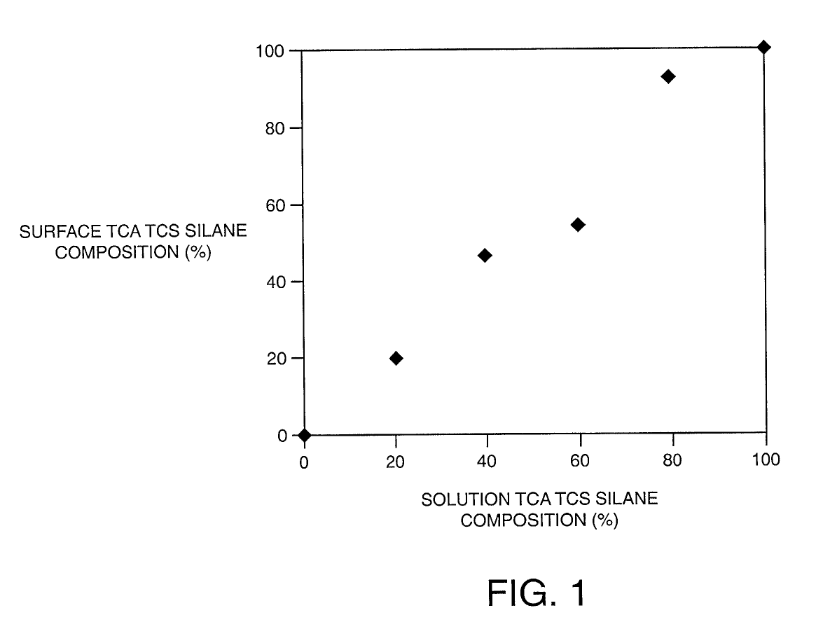

X-Ray Photoelectron Spectroscopy, (XPS) was utilized to characterize the fluorine signal of the mixed methyl and trichloroacetyl (TCA) terminated SAMs, and the results are presented in FIG. 1. The surface compositions of TCA groups with the proportion of TCA undecyl TCS in the solution mixture were correlated. From FIG. 1 it is evident that the relationship between the solution and surface composition of TFA groups is close to linear. Though similar to the plot of OH group solution versus surface compositions in mixed alkanethiol SAM systems, the increased linearity strongly suggests that the chemisorption reaction kinetics of the methyl and TCA terminated TCS with a silicon surface are very similar.

In one embodiment, mixed methyl and hydroxy terminated TCS formed SAMs with varied hydroxy group densities. However, since the TCS group is highly reactive towards any hydroxy groups TCA and TFA protecting groups can be to prevent side reactions from occurring. Clean silicon surfaces were immersed in xylene solutions containing mixtures of octyl and TFA or TCA TCS species, at a concentration of 1 μl/ml for 1 hour, under ambient conditions. Although TCS is very hygroscopic, SAM formed readily in ambient air as long as the solvent was dry. The reaction mechanism was also hydrolysis of the TCS groups to silanol groups, possibly with residual water adsorbed on the silicon surfaces as well as traces in the organic solvent. The silanols would then quickly react with hydroxy groups on the native oxide surfaces, produced in the reducing environment of the base bath used to clean the silicon chips. Subsequent immersion in a basic solution would remove the protecting groups to expose the hydroxy groups. Both TFA and TCA TCS species can produce the hydroxy surface groups, but the TFA protecting group is less stable compared to TCA groups.

IV. Nucleic Acids

After derivatization of the substrate, the derivatized surface may be contacted with biopolymeric compound, such as an oligonucleotide.

Oligonucleotide portions of the invention may comprise any polymeric compound capable of specifically binding to a target polynucleotide by way of a regular pattern of monomer-to-nucleoside interactions, such as Watson-Crick type of base pairing, Hoogsteen or reverse Hoogsteen types of base pairing, or the like. The oligonucleotide portion may be modified to enhance its physical properties.

Oligonucleotides of the present invention may include non-phosphate internucleosidic linkages. Many such linkages are known in the art, e.g., phosphorothioate: Zon and Geiser, Anti-Cancer Drug Design, 6:539-568 (1991); Stec et al., U.S. Pat. No. 5,151,510; Hirschbein, U.S. Pat. No. 5,166,387; Bergot, U.S. Pat. No. 5,183,885; phosphorodithioates: Marshall et al., Science, 259:1564-1570 (1993); Caruthers and Nielsen, International application PCT/US89/02293; phosphoramidates, e.g., —OP(═O)(NR₁ R₂)—O— with R₁ and R₂ hydrogen or C₁-C₃ alkyl; Jager et al., Biochemistry, 27:7237-7246 (1988); Froehler et al., International application PCT/US90/03138; peptide nucleic acids: Nielsen et al., Anti-Cancer Drug Design, 8:53-63 (1993), International application PCT/EP92/01220; methylphosphonates: Miller et al., U.S. Pat. No. 4,507,433, Ts'o et al., U.S. Pat. No. 4,469,863; Miller et al., U.S. Pat. No. 4,757,055; and P-chiral linkages of various types, especially phosphorothioates, Stec et al., European patent application 506,242 (1992) and Lesnikowski, Bioorganic Chemistry, 21:127-155 (1993). Additional non-phosphate linkages include phosphoroselenoate, phosphorodiselenoate, phosphoroanilothioate, phosphoranilidate, alkylphosphotriester such as methyl- and ethylphosphotriester, carbonates such as carboxymethyl ester, carbamate, morpholino carbamate, 3′-thioformacetal, silyl such as dialkyl (C₁-C₆)— or diphenylsilyl, sulfamate ester, and the like. Such linkages and methods for introducing them into oligonucleotides are described in many references, e.g., reviewed generally by Peyman and Ulmann, Chemical Reviews 90:543-584 (1990); Milligan et al., J. Med. Chem., 36:1923-1937 (1993); Matteucci et al., International application PCT/US91/06855.

Preferably, phosphorus analogs of the phosphodiester linkage employed in the compounds of the invention, are selected from phosphorothioate, phosphorodithioate, phosphoramidate, or methylphosphonate.

Phosphorothioate oligonucleotides contain a sulfur-for-oxygen substitution in the internucleotide phosphodiester bond. Phosphorothioate oligonucleotides are capable of effective hybridization for duplex formation, and retain the water solubility of a charged phosphate analogue.

It is understood that in addition to the phosphodiester and other preferred linkage groups, oligonucleotides may comprise additional modifications, e.g., boronated bases, Spielvogel et al., U.S. Pat. No. 5,130,302; cholesterol moieties, Shea et al., Nucleic Acids Research, 18:3777-3783 (1990) or Letsinger et al., Proc. Natl. Acad. Sci., 86:6553-6556 (1989); and 5-propynyl modification of pyrimidines, Froehler et al., Tetrahedron Lett., 33:5307-5310 (1992).

Preferably, oligonucleotide portions of the invention are synthesized by conventional means on commercially available automated DNA synthesizers, e.g., an Applied Biosystems (Foster City, Calif.) model 380B, 392 or 394 DNA/RNA synthesizer. Preferably, phosphoramidite chemistry is employed, e.g., as disclosed in the following references: Beaucage and Iyer, Tetrahedron, 48:2223-2311 (1992); Molko et al., U.S. Pat. No. 4,980,460; Koster et al., U.S. Pat. No. 4,725,677; Caruthers et al., U.S. Pat. Nos. 4,415,732; 4,458,066; and 4,973,679.

In embodiments where triplex formation is desired, there are constraints on the selection of target sequences. Generally, third strand association via Hoogsteen type of binding is most stable along homopyrimidine-homopurine tracks in a double stranded target. Usually, base triplets form in T−A*T or C−G*C motifs (where “−” indicates Watson-Crick pairing and “*” indicates Hoogsteen type of binding); however, other motifs are also possible. For example, Hoogsteen base pairing permits parallel and antiparallel orientations between the third strand (the Hoogsteen strand) and the purine-rich strand of the duplex to which the third strand binds, depending on conditions and the composition of the strands. There is extensive guidance in the literature for selecting appropriate sequences, orientation, conditions, nucleoside type (e.g., whether ribose or deoxyribose nucleosides are employed), base modifications (e.g., methylated cytosine, and the like) in order to maximize, or otherwise regulate, triplex stability as desired in particular embodiments, e.g., Roberts et al., Proc. Natl. Acad. Sci., 88:9397-9401 (1991); Roberts et al., Science, 58:1463-1466 (1992); Distefano et al., Proc. Natl. Acad. Sci., 90:1179-1183 (1993); Mergny et al., Biochemistry, 30:9791-9798 (1992); Cheng et al., J. Am. Chem. Soc., 114:4465-4474 (1992); Beal and Dervan, Nucleic Acids Research, 20:2773-2776 (1992); Beal and Dervan, J. Am. Chem. Soc., 114:4976-4982; Giovannangeli et al., Proc. Natl. Acad. Sci., 89:8631-8635 (1992); Moser and Dervan, Science, 238:645-650 (1987); McShan et al., J. Biol. Chem., 267:5712-5721 (1992); Yoon et al., Proc. Natl. Acad. Sci., 89:3840-3844 (1992); and Blume et al., Nucleic Acids Research, 20:1777-1784 (1992).

The length of the oligonucleotide moieties may be sufficiently large to ensure that specific binding will take place only with the desired target polynucleotide and not at other adventitious sites, as explained in many references, e.g., Rosenberg et al., International application PCT/US92/05305; or Szostak et al., Meth. Enzymol, 68:419-429 (1979). The desired length is determined by several factors, including the inconvenience and expense of synthesizing and purifying oligomers greater than about 30-40 nucleotides in length, the greater tolerance of longer oligonucleotides for mismatches than shorter oligonucleotides, whether modifications to enhance binding or specificity are present, whether duplex or triplex binding is desired, and the like. Usually, oligonucleotides useful in the invention have lengths in the range of about 12 to 60 nucleotides. More preferably, oligonucleotides of the invention have lengths in the range of about 15 to 40 nucleotides; and most preferably, they have lengths in the range of about 18 to 30 nucleotides.

In general, the oligonucleotides used in the practice of the present invention will have a sequence which is completely complementary to a selected polynucleotide or portion thereof. Absolute complementarity is not, however, required, particularly in larger oligomers. Thus, reference herein to a “nucleotide sequence complementary to” a target polynucleotide does not necessarily mean a sequence having 100% complementarity with the target segment. In general, any oligonucleotide having sufficient complementarity to form a stable duplex with the target (e.g., an oncogene mRNA), that is, an oligonucleotide which is “hybridizable”, is suitable. In preferred embodiments, however, a complementary sequence is 100% complementary to a target nucleotide sequence. Stable duplex formation depends on the sequence and length of the hybridizing oligonucleotide and the degree of complementarity with the target polynucleotide. Generally, the larger the hybridizing oligomer, the more mismatches may be tolerated. More than one mismatch may not be suitable for oligomers of less than about 21 nucleotides. One skilled in the art may readily determine the degree of mismatching which may be tolerated between any given oligomer and the target sequence, based upon the melting point, and therefore the thermal stability, of the resulting duplex.

The thermal stability of hybrids formed by the oligonucleotides of the invention may be determined by way of melting, or strand dissociation, curves. The temperature of fifty percent strand dissociation is taken as the melting temperature, Tₘ, which, in turn, provides a convenient measure of stability. Tₘ measurements are typically carried out in a saline solution at neutral pH with target and oligonucleotide concentrations at between about 1.0-2.0 μM. Typical conditions are as follows: 150 mM NaCl and 10 mM MgCl₂ in a 10 mM sodium phosphate buffer (pH 7.0) or in a 10 mM Tris-HCl buffer (pH 7.0). Data for melting curves are accumulated by heating a sample of the oligonucleotide/target polynucleotide complex from room temperature to about 85° C. As the temperature of the sample increases, absorbance of 260 nm light is monitored at 1° C. intervals, e.g., using a Cary (Australia) model 1E or a Hewlett-Packard (Palo Alto, Calif.) model HP 8459 UV/VIS spectrophotometer and model HP 89100A temperature controller, or like instruments. Such techniques provide a convenient means for measuring and comparing the binding strengths of oligonucleotides of different lengths and compositions.

Crosslinking agents which can be readily incorporated into nucleic acid sequences, such as psoralen C6 phosphoramidite and psoralen C2 phosphoramidite, available from Glen Research, in Sterling, Va., can be used to easily prepare nucleic acid sequences which can be crosslinked after hybridization.

In one embodiment, oligonucleotide surface arrays are created by direct stepwise oligonucleotide synthesis onto a flat silicon surface chemically derivatized by a SAM of end-functionalized silanes. By manipulating the density of reactive sites on the SAM surface, the oligonucleotide surface density is controlled, and ascertained as to the stepwise reaction efficiency, and the hybridization yield to complementary solution-phase oligonucleotide chains. In addition, a sensitive surface characterization technique, XPS, was utilized with specially labeled nucleotide bases to quantify the surface density of the reactive sites on the silane SAM surface, and the surface oligo density under various reaction conditions, and utilized the results to determine the optimal conditions that maximized the sequence fidelity of the surface oligo chains. By manipulating the silane film compositions using a mixed SAM methodology, surface oligo films with a controlled surface density were created, which were used to systematically study the steric and the hybridization yield with labeled complementary oligos in solution.

V. Capture Oligonucleotides

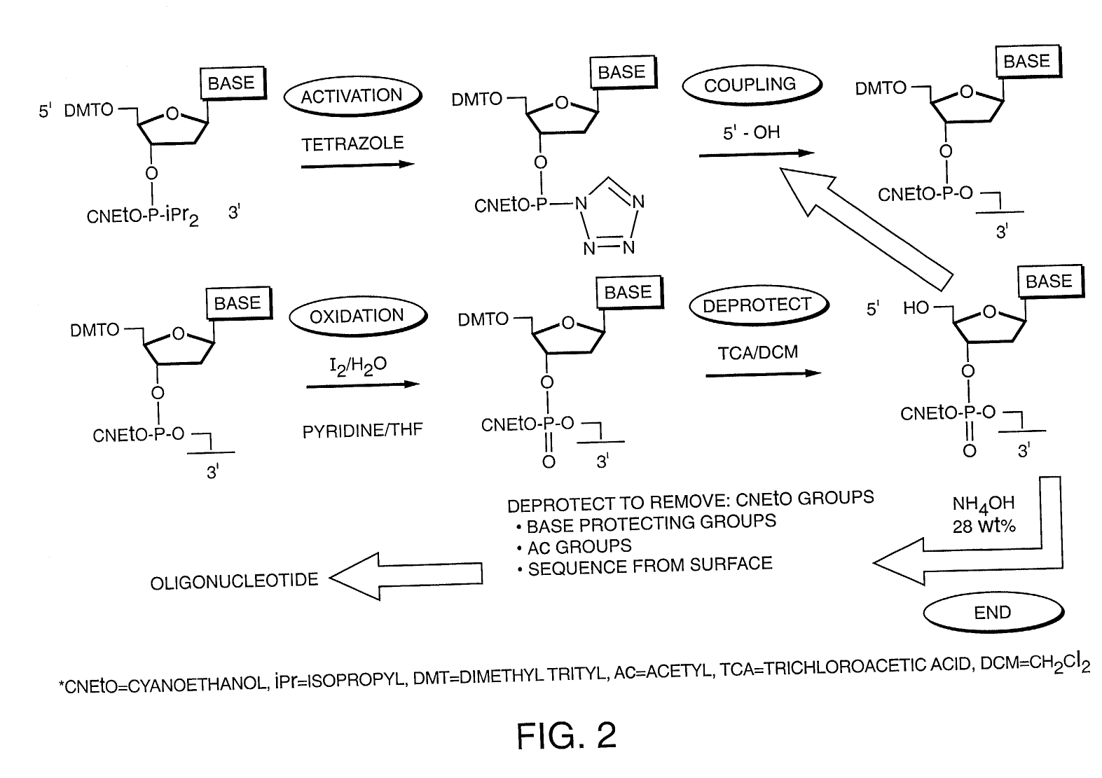

After producing and characterizing the various hydroxyl surface films on silicon surfaces, oligonucleotide chains can be synthesized on these reactive surfaces by standard phosphoramidite chemistry using an automated oligo synthesizer. The synthesis cycle consists of four basic steps, detritylation, coupling, capping and oxidation, and the reaction chemistry is diagrammed in detail in FIG. 2. First, the acid labile dimethyl trityl (DMT) protecting group is removed from the 5′ end of an immobilized oligo with 3wt % trichloroacetic acid in dimethylformamide (DMF), to expose the reactive hydroxyl group. Next, a phosphoramidite in solution is activated on the 3′ end with tetrazole, and the incoming activated tetrazoyl phosphoramidite is coupled to the 5′ hydroxyl group of the immobilized oligo. Then an unreacted 5′ end hydroxyl groups on the surface are capped with acetic anhydride, activated with n-methyl imidazole to produce an acetylating reagent, to prevent unwanted side reactions that would result in incorrect sequences and shorter chain lengths. Finally, a 0.1 M iodine in a solution mixture of water and THF is used to oxidize the chemically unstable phosphite triester group to form a stable pentavalent phosphate triester entity, which is not susceptible cleavage by acidic agents such as the trichloroacetic acid/DCM solution subsequently used to deprotect the 5′ DMT group of the coupled nucleotide. The oxidation reaction completes the oligo addition cycle, and with each subsequent cycle another oligo is added stepwise to the surface-immobilized oligo chain. Upon completion the oligos are exposed to 28wt % NH₄OH_{(aq)}, which cleaved the oligo chain from the surface, removed the cyanoethyl group from the phosphate triester to form an electronegative P—O⁻ group, and also removed base protecting groups from the nucleotide bases to expose the amine groups necessary for the formation of hydrogen bonds during DNA hybridization.

In one embodiment, a photoactivated crosslinker molecule such as psoralen may be attached to the 5′ end of the surface-attached oligonucleotide. Photoactivated crosslinker molecules form a covalent bond with a specific other molecule or small group of molecules via a cycloaddition or photosubstitution reaction. These compounds include thymidine which reacts with thymidine to form a covalent link. Other compounds of this nature include but are not limited to coumorin, substituted coumarins, furocoumarin, isocoumarin, bis-coumarin, psoralen, psoralen derivatives, 8-methoxypsoralen, aminomethyl trimethyl psoralen, quinones, pyrones, α- and β-unsaturated acids, acid derivatives, esters, ketones, nitriles and azidos.

In one embodiment, the instant invention may require that the oligonucleotide chains are chemically stable and remain attached to the surface under a variety of conditions. In a particular embodiment, hydroxy terminated silanes are used because they form a phosphate bond with the nucleotides, which is the same bond present in the oligo and dsDNA backbones.

In one embodiment, deprotection chemistry is employed. The protecting groups are fairly stable and only removed by exposure to a strong ammoniolysis agent such as NH₄OH_{(aq)} at elevated temperatures (80° C.) for 1 hour or at room temperature for long periods of time (4-16 hours). However, the NH₄OH_{(aq)} was also a strong base that readily removed the synthesized oligo chains from the surface. We conducted studies on the mechanism for base-induced desorption using oligo surfaces and iodinated silane SAMs, with XPS data from iodine labeled 1^{st} nucleotide surfaces exposed to 28 wt % NH₄OH_{(aq)} for various time periods. Even at room temperature approximately 50% of the surface-attached oligos were removed after 15 minutes of immersion, and the attached oligonucleotides were completely removed after 24 hours immersion. Thus the majority of the surface-immobilized oligonucleotide chains would be removed into solution even before they are effectively deprotected and useful for hybridization experiments.

In one embodiment, the oligonucleotide synthesis strategy may be to produce oligonucleotide chains with optimal coupling efficiency and sequence fidelity, and choose deprotection protocols that preserved the synthesized oligo chains on the surface, and control the oligo surface density strictly by modifying the silicon substrate with the appropriate silane SAM films to create the proper 1^{st} nucleotide surface density. In one embodiment, UltraMILD® phosphoramidites (Glen Research), are used. The UltraMILD® groups are more labile compared to the regular base protecting groups, and are removed by a variety of mild chemical agents at room temperature. Next, four alternative deprotection protocols were studied concurrently using surface-immobilized oligo chains synthesized with UltraMILD® phosphoramidites. Either 0.05 M potassium carbonated (K₂CO₃) in anhydrous methanol (MeOH), or a 28 wt % NH₄OH_{(aq)} solution was used to remove the base protecting groups. Next, either 20 wt % piperidine in dimethyl formamide (DMF), or 28 wt % NH₄OH was used to deprotect the cyanoethyl groups and create the hydrophilic P—O⁻ groups. All deprotection reactions were carried out at ambient temperature and pressure. Both XPS and ellipsometry were used to determine the amount of surface-immobilized oligo chains (labeled with iodinated U) remaining after treatment with the various deprotecting protocols, by comparing the data values for the oligo surfaces before, and after chemical deprotection.

In addition, hybridization experiments were performed using ³²P-labeled complementary oligo sequences in solution to challenge the surface-immobilized oligo sequence deprotected by the various chemical agents. The results of the tests conducted on the various mild deprotection protocols are presented in Table 2. The data indicate that oligo surfaces synthesized on the OH-terminated surfaces had higher hybridization signals compared to oligo surfaces synthesized on the glycol-terminated surfaces. Table 2 shows a comparison of the various deprotection protocols, including traditional agents used for PCR (Polymerase chain reaction) primer synthesis and the mild agents used to remove the UltraMILD® protecting groups. The remaining oligo coverage (%) is determined by comparing the oligo surfaces before and after deprotection, using XPS with iodinated Uracil, and by ellipsometry. The results show that mild deprotection does not remove the surface oligo chains. Hybridization reactions with radiolabeled complementary solution oligos were used to determine whether the deprotected surface oligo chains are functional.

TABLE 2

| | Coverage | | |
|---|---|---|---|
| Deprotection | Remaining | ³²P signal on glycol | ³²P signal on OH |
| Protocol | (%) | linked oligo (cpm) | linked oligo (cpm) |
| K₂CO₃/MeOH & | 100 | 4276 | 13638 |
| Piperidine/DMF | | | |
| NH₄OH | 27 | 2220 | 6384 |
| Piperidine/DMF | | | |
| K₂CO₃/MeOH & | 45 | 294 | 2082 |
| NH₄OH | | | |
| NH₄OH | 5 | 198 | 2548 |

*K₂CO₃/MeOH = 0.05M K₂CO₃ in anhydrous methanol for 8 hrs, Piperidine/DMF = 20 wt % piperidine in dimethyl formamide for 4 hrs, NH₄OH = 28 wt % NH₄OH_{(aq)} for 1-3 hrs

Surface-immobilized oligos of various lengths (10-20 mers) and discrete sequences were synthesized on silicon surfaces with different hydroxyl group densities, to study how the surface density of the 1^{st} nucleotide added to the hydroxyl surface film (as previously determined by iodine-labeled U phosphoramidites and XPS characterization), and the synthesis reaction conditions, could affect both the sequence fidelity of the surface-immobilized oligos, as well as the surface density of full length oligo chains available for hybridization. The major factor thought to influence oligo surface density and sequence fidelity during synthesis was the coupling of the incoming activated phosphoramidite in solution to the reactive 5′ hydroxy end of the surface-attached oligo chain. The stepwise efficiency of this coupling reaction is critical to the amount of full-length oligo chains produced upon completion of the oligo synthesis.

In one embodiment, oligonucleotide surfaces are created using standard cyanoethyl cycles with a coupling reaction time of 25 seconds. A variety of silane SAMs with different hydroxy group density, including GOPS silane, OH silane and mixed TCS surfaces was used to study the effect of the steric hindrance on oligo coupling efficiency. The proportion of full-length oligos synthesized is dependent not only on the coupling reaction efficiency, but also on the capping efficiency. The unreacted 5′ OH groups on the surface-immobilized oligo chains must be effectively capped with acetyl protecting groups to prevent them from reacting with activated phosphoramidites in solution during subsequent coupling reaction cycles.

In one embodiment, oligonucleotide surfaces are stepwise synthesized with a coverage density of up to 8×10⁻¹¹ moles/cm². This surface oligo density was at least one order of magnitude higher compared to flat 2-D oligo surfaces produced by covalent attachment of an oligo with an amine, thiol, or biotin linker to an activated surface, regardless of the surface chemistry.

In a preferred embodiment, the density of the oligos is between 1×10⁻¹¹ and 12×10⁻¹² moles/cm². In a more preferred embodiment, the density of the oligos is between 2×10⁻¹¹ and 6×10⁻¹² moles/cm². In even more preferred embodiment, the density of the oligos is between 3×10⁻¹¹ moles/cm² and 6×10⁻¹² moles/cm².

In one embodiment, the oligonucleotide surface density is controlled by manipulating the composition and chemical moeties of the silane SAM formed on the silicon surface.

In one embodiment, oligonucleotides are presynthesized and microspotted onto the derivatized surface. In another embodiment, the oligonucleotides are synthesized on the surface using microlithography, for example disclosed in U.S. Pat. No. 6,165,717 and references therein.

In one embodiment, different oligonucleotides may be positioned on the surface. In another embodiment, all the oligonucleotides differ on the surface.

Oligonucleotide chips stored dry at 4° C. for over three months did not demonstrate any significant decrease in the amount of DNA hybridized to the surface, indicating that the oligonucleotide chips are chemical stable and can be stored for long periods of time prior to use. Tests on the recycle ability of the oligonucleotide surfaces were performed by heating an oligo chip with ³²P-labeled complementary oligonucleotide hybridized onto the surface, to 90° C. in 0.1 wt % SDS aqueous solution for 30 minutes, to remove the ³²P-labeled oligonucleotide chains. Then the oligo chip was rinsed with deionized water and washed again in 90° C. 0.1 wt % SDS aqueous solution for 30 minutes, after which only background radiation was detected, indicating complete removal of the ³²P-labeled oligo from the surface. Next, the oligonucleotide chip was immersed in a fresh solution of complementary, radiolabeled oligonucleotide chains, and the amount of oligo hybridized to the oligonucleotide chip was measured by liquid scintillation counting. For all cycles complete desorption of labeled oligonucleotide was observed after heating, and there was no decrease in ³²P radiation signal during the subsequent hybridization reactions. This is expected since the oligonucleotide surfaces are covalently bound to the silicon substrate, and the nucleotide bases are chemically stable in the absence of enzymes. Further, the results demonstrate the thermal stability and recycle capabilities of the surface immobilized oligo chips.

In one embodiment, the ability to produce oligonucleotide chips on silicon surfaces with high sequence fidelity, thermal stability and with consistent, controllable surface density may be the basis for the synthesis of oligonucleotide chips with optimal hybridization performance, to immobilize oligonucleotide or DNA chains to a flat silicon surface. The oligonucleotide surfaces themselves could be as probes to detect complementary oligonucleotides are ssDNA in solution, or as the platform to immobilize a longer chain dsDNA to form a surface probe using ssDNA.

V. Conjugates

The present invention also provides a conjugate. The conjugate comprises a pairing oligonucleotide and a target moiety. The conjugate may also optionally comprise a crosslinking moiety attached to the pairing oligonucleotide and/or a linking group connecting the pairing oligonucleotide and the target moiety.

The pairing oligonucleotide is a single stranded nucleic acid molecule which mediates attachment of the target moiety to the solid support via interaction with the capture oligonucleotide. The pairing oligonucleotide may be of any desired length that is suitable for the experiment being performed. For example, a short pairing oligonucleotide may be used when low complexity arrays are being developed while longer pairing oligonucleotides may be needed for developing very complex arrays. As a particular example, a pairing oligonucleotide of 4 nucleotides in length would provide a total of 4⁴ or 256 different sequences which could specifically hybridize to the 256 complementary capture oligonucleotides. A pairing oligonucleotide of 10 nucleotides would increase the number of sequences to 4¹⁰ or 1,048,576 therefore providing a much higher level of complexity. Pairing oligonucleotides preferably comprise between 2 and 200 nucleotides, more preferably between 5 and 50 nucleotides and most preferably between 5 and 20 nucleotides.

The target moiety is a molecule which may be contacted with a test sample in order to determine if an interaction is occurring between the target moiety and a molecule in the test sample. Examples of target moieties that may be used in accord with the invention include, but are not restricted to, agonists and antagonists for cell membrane receptors, toxins and venoms, viral epitopes, hormones, opiates, steroids, hormone receptors, peptides, enzymes, enzyme substrates, cofactors, drugs, lectins, sugars, oligonucleotides, oligosaccharides, proteins, peptides, antibodies, antibody fragments and small molecules. Oligonucleotide target moieties may be RNA or DNA, may be single stranded, double standed or triple standed and may be linear or contain elements of secondary and/or tertiary structure. In a preferred embodiment, the target moiety is a double stranded nucleic acid molecule which may be converted into a single stranded oligonucleotide via separation of the two strands. A nucleic acid targeting moiety may be of any length, but preferable comprises from 5 to 5,000 nucleotides, more preferably from 20 to 2,000 nucleotides and most preferably from 50 to 1,000 nucleotides.

The invention also provides libraries of conjugates. In one embodiment, a library of targeting moieties are attached to a common pairing oligonucleotide. In another embodiment, a library of targeting moieties are attached to a library of pairing oligonucleotides.

The joining of a target moiety to the pairing oligonucleotide may be effected by any means which produces a link between two or more constituents that is sufficiently stable to withstand the conditions used and which does not alter the function, e.g., binding, of either constituent. For example, the attachment of the pairing oligonucleotide to the target moiety preferably is able to withstand moderate to extreme conditions of heat, salt and/or pH such as may be used for hybridization or denaturation of nucleic acid strands. Preferably, the link between the pairing oligonucleotide and the target moiety is covalent.

The pairing oligonucleotide and the target moiety may be attached directly by linking a functional group present at the termini, or any other location, of one molecule to a functional group at the termini, or any other location, of the other molecule. Alternatively, the pairing oligonucleotide and the target moiety may be attached indirectly via a linker group. Suitable methods for linking the various portions are discussed below.

The target moiety may attached to either the 5′ or the 3′ end of the pairing oligonucleotide. Additionally, when the target moiety is itself a nucleic acid it may be attached through its 5′ or 3′ to either end of the pairing oligonucleotide. In a preferred embodiment, the target moiety is attached to the pairing oligonucleotide at either the 5′ or 3′ terminus of the oligonucleotide so as to provide end immobilized target moieties when the conjugate is attached to a solid support.

Numerous chemical crosslinking methods are known and potentially applicable for conjugating the various portions of the instant conjugates. Many known chemical crosslinking methods are non-specific, i.e., they do not direct the point of coupling to any particular site on the polypeptide, polynucleotide, or other molecule. As a result, use of non-specific crosslinking agents may attack functional sites or sterically block active sites, rendering the conjugated moieties resistant to binding to their desired binding partners.

Many methods for linking compounds, such as proteins, labels, oligonucleotides and other chemical entities, to nucleotides are known in the art. For example, substituents have been attached to the 5′ end of preconstructed oligonucleotides using amidite or H-phosphonate chemistry, as described by Ogilvie, K. K., et al., Pure and Appl Chem (1987) 59:325, and by Froehler, B. C., Nucleic Acids Res (1986) 14:5399. Substituents have also been attached to the 3′ end of oligomers, as described by Asseline, U., et al., Tet Lett (1989) 30:2521. This last method utilizes 2,2′-dithioethanol attached to a solid support to displace diisopropylamine from a 3′ phosphonate bearing the acridine moiety and is subsequently deleted after oxidation of the phosphorus. Other substituents have been bound to the 3′ end of oligomers by alternate methods, including polylysine (Bayard, B., et al., Biochemistry (1986) 25:3730; Lemaitre, M., et al., Nucleosides and Nucleotides (1987) 6:311) and, in addition, disulfides have been used to attach various groups to the 3′ terminus, as described by Zuckerman, R., et al., Nucleic Acids Res (1987) 15:5305. It is known that oligonucleotides which are substituted at the 3′ end show increased stability and increased resistance to degradation by exonucleases (Lancelot, G., et al., Biochemistry (1985) 24:2521; Asseline, U., et al., Proc Natl Acad Sci USA (1984) 81:3297). Other examples of suitable cross-linking reagents include n-γ-maleimidobutyryloxy-succinimide ester (“GMBS”) and sulfo-GMBS. Additional methods of attaching non-nucleotide entities to oligonucleotides are discussed in U.S. Pat. Nos. 5,321,131 and 5,414,077.

Alternatively, the pairing oligonucleotide and/or target oligonucleotide moiety may include one or more modified nucleotides having a group attached via a linker arm to the base. For example, Langer et al. (Proc. Natl. Acad. Sci. U.S.A., 78(11):6633-6637, 1981) describes the attachment of biotin to the C-5 position of dUTP by an allylamine linker arm. The attachment of biotin and other groups to the 5-position of pyrimidines via a linker arm is also discussed in U.S. Pat. No. 4,711,955. Nucleotides labeled via a linker arm attached to the 5- or other positions of pyrimidines are also suggested in U.S. Pat. No. 4,948,882. Bisulfite-catalyzed transamination of the N⁴-position of cytosine with bifunctional amines is described by Schulman et al. (Nucleic Acids Research, 9(5): 1203-1217, 1981) and Draper et al (Biochemistry, 19: 1774-1781, 1980). By this method, chemical entities are attached via linker arms to cytidine or cytidine-containing polynucleotides. The attachment of biotin to the N⁴-position of cytidine is disclosed in U.S. Pat. No. 4,828,979, and the linking of moieties to cytidine at the N⁴-position is also set forth in U.S. Pat. Nos. 5,013,831 and 5,241,060. U.S. Pat. No. 5,407,801 describes the preparation of an oligonucleotide triplex wherein a linker arm is conjugated to deoxycytidine via bisulfite-catalyzed transamination. The linker arms include an aminoalkyl or carboxyalkyl linker arm. U.S. Pat. No. 5,405,950 describes cytidine analogs in which a linker arm is attached to the N⁴-position of the cytosine base.

Numerous crosslinking reagents, including the ones discussed above, are commercially available. Detailed instructions for their use are readily available from the commercial suppliers. A general reference on protein crosslinking and conjugate preparation is: S. S. Wong, Chemistry of Protein Conjugation and Cross-Linking, CRC Press (1991).

Chemical crosslinking may include the use of spacer arms. Spacer arms provide intramolecular flexibility or adjust intramolecular distances between conjugated moieties and thereby may help preserve biological activity. A spacer arm may be in the form of a polypeptide moiety comprising spacer amino acids. Alternatively, a spacer arm may be part of the crosslinking reagent, such as in “long-chain SPDP” (Pierce Chem. Co., Rockford, Ill., cat. No. 21651 H).

A preferred approach to increasing coupling specificity in the practice of this invention is direct chemical coupling to a functional group found only once or a few times in one or both of the molecules to be conjugated. For example, in many proteins, cysteine, which is the only protein amino acid containing a thiol group, occurs only a few times. Also, for example, if a polypeptide contains no lysine residues, a cross-linking reagent specific for primary amines will be selective for the amino terminus of that polypeptide. Successful utilization of this approach to increase coupling specificity requires that the molecule have the suitable reactive residues in areas of the molecule that may be altered without loss of the molecule's biological activity.

Cysteine residues may be replaced when they occur in parts of a polypeptide sequence where their participation in a cross-linking reaction would likely interfere with biological activity. When a cysteine residue is replaced, it is typically desirable to minimize resulting changes in polypeptide folding. Changes in polypeptide folding are minimized when the replacement is chemically and sterically similar to cysteine. For these reasons, serine is preferred as a replacement for cysteine. Alternatively, a cysteine residue may be introduced into a polypeptide's amino acid sequence for cross-linking purposes. When a cysteine residue is introduced, introduction at or near the amino or carboxy terminus is preferred. Conventional methods are available for such amino acid sequence modifications, whether the polypeptide of interest is produced by chemical synthesis or expression of recombinant DNA.

Coupling of the two constituents can be accomplished via a coupling or conjugating agent. There are several intermolecular crosslinking reagents which can be utilized (see, for example, Means, G. E. and Feeney, R. E., Chemical Modification of Proteins, Holden-Day, 1974, pp. 39-43). Among these reagents are, for example, J-succinimidyl 3-(2-pyridyldithio) propionate (SPDP) or N,N′-(1,3-phenylene) bismaleimide (both of which are highly specific for sulfhydryl groups and form irreversible linkages); N,N′-ethylene-bis-(iodoacetamide) or other such reagent having 6 to 11 carbon methylene bridges (which relatively specific for sulfhydryl groups); and 1,5-difluoro-2,4-dinitrobenzene (which forms irreversible linkages with amino and tyrosine groups). Other crosslinking reagents useful for this purpose include: p,p′-difluoro-m,m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine).

Cross-linking reagents may be homobifunctional, i.e., having two functional groups that undergo the same reaction. A preferred homobifunctional crosslinking reagent is bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible attachment of a polypeptide to another molecule that contains cysteine residues.

Crosslinking reagents may also be heterobifunctional. Heterobifunctional crosslinking agents have two different functional groups, for example an amine-reactive group and a thiol-reactive group, that will cross-link two molecules having free amines and thiols, respectively. Heterobifunctional crosslinkers provide the ability to design more specific coupling methods for conjugating two chemical entities, thereby reducing the occurrences of unwanted side reactions such as homo-protein polymers. A wide variety of heterobifunctional crosslinkers are known in the art. Examples of heterobifunctional crosslinking agents are succinimidyl 4-(N-maleimidomethyl)-cyclohexane-1-carboxylate (SMCC), N-succinimidyl (4-iodoacetyl) aminobenzoate (SIAB), 1-ethyl-3-(3-dimethylaminopropyl) carbodiimide hydrochloride (EDC); 4-succinimidyloxycarbonyl-a-methyl-a-(2-pyridyldithio)-toluene (SMPT), N-succinimidyl 3-(2-pyridyldithio) propionate (SPDP), succinimidyl 6-[3-(2-pyridyldithio) propionate] hexanoate (LC-SPDP)succinimidyl 4-(N-maleimidomethyl)-cyclohexane-1-carboxylate (“SMCC”), m-maleimidobenzoyl-N-hydroxysuccinimide ester (“MBS”), and succinimide 4-(p-maleimidophenyl)butyrate (“SMPB”), an extended chain analog of MBS. The succinimidyl group of these crosslinkers reacts with a primary amine, and the thiol-reactive maleimide forms a covalent bond with the thiol of a cysteine residue.

Crosslinking reagents often have low solubility in water. A hydrophilic moiety, such as a sulfonate group, may be added to the cross-linking reagent to improve its water solubility. Sulfo-MBS and sulfo-SMCC are examples of crosslinking reagents modified for water solubility.

Another reactive group useful as part of a heterobifunctional crosslinker is a thiol reactive group. Common thiol-reactive groups include maleimides, halogens, and pyridyl disulfides. Maleimides react specifically with free sulfhydryls (cysteine residues) in minutes, under slightly acidic to neutral (pH 6.5-7.5) conditions. Haloalkyl groups (e.g., iodoacetyl functions) react with thiol groups at physiological pH's. Both of these reactive groups result in the formation of stable thioether bonds.

In addition to the heterobifunctional crosslinkers, there exist a number of other crosslinking agents including homobifunctional and photoreactive crosslinkers. Disuccinimidyl suberate (DSS), bismaleimidohexane (BMH) and dimethylpimelimidate-2 HCl (DMP) are examples of useful homobifunctional crosslinking agents, and bis-[β-(4-azidosalicylamido)ethyl]disulfide (BASED) and N-succinimidyl-6(4′-azido-2′-nitrophenyl-amino)hexanoate (SANPAH) are examples of useful photoreactive crosslinkers for use in this invention. For a recent review of protein coupling techniques, see Means et al. (1990) Bioconjugate Chemistry 1:2-12, incorporated by reference herein.

Both oligonucleotides and oligopeptides may be readily prepared by automated synthesis using conventional synthesizers. Oligonucleotide synthesis is described above. For peptide synthesis, commercially available amino acids having chemically removable protecting groups are used, for example FMOC-amino acids. After exchange of the protecting groups, the coupling steps can be carried out using BOP/HOBt activation and coupling methods. Those of skill in the art will understand that other coupling methods as well as other amino acid monomers having chemically removable protecting groups can be used in the present invention.

The conjugate may also comprise a crosslinking reagent attached to the pairing oligonucleotide which can covalently attach the pairing oligonucleotide to the capture oligonucleotide. Any crosslinking agent capable of covalently attaching two oligonucleotides may be used. Various descriptions of suitable crosslinking agents are described in above. In a preferred embodiment, the crosslinking agent is psoralen. Psoralen is a photoactivated crosslinking molecule with a rigid, flat structure that readily intercalates within the dsDNA double helix, preferable between an AT sequence. Both the furan and pyrone functional groups of the psoralen compound are photolyzed with long wavelength UV light (365 nm) to form covalent bonds with particular nucleotide bases. The furan side is 4 times more reactive than the pyrone side an overwhelmingly favors reacting with T nucleotides, though they have limited reactivity also with C and U nucleotides. The crosslinking can result in the formation of monoadducts or diadducts by varying the wavelength and intensity of the UV illumination source. Finally, the crosslinking is reversible by exposure to short wavelength (254 nm) UV light, which appears to cleave the covalent bonds. Psoralen and methylated psoralen derivatives have been used to crosslink two complementary ssDNA strands of a dsDNA duplex together. Psoralen, psoralen derivatives and special phosphoramidites with 5′ psoralen linkers are commercially available (Glen Research).

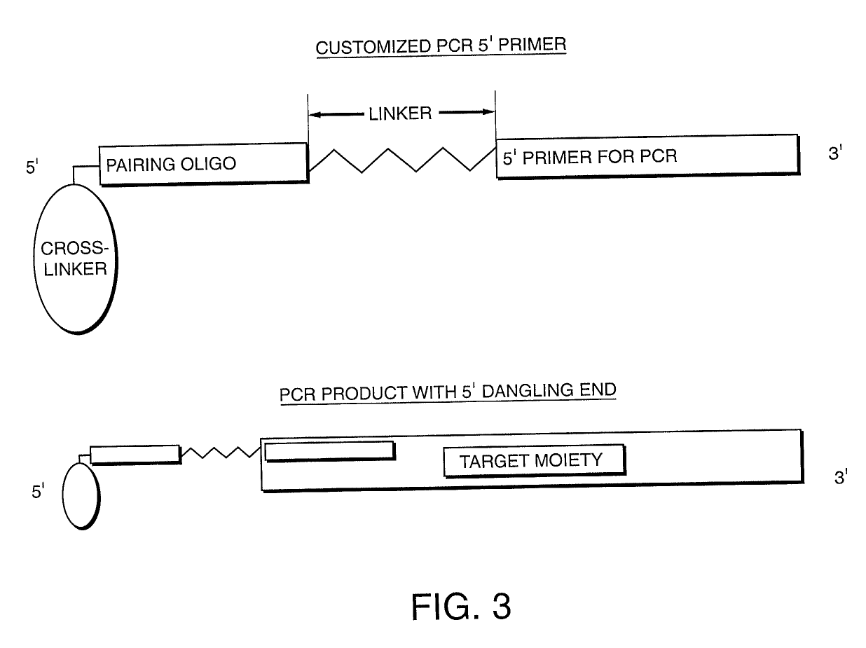

In a preferred embodiment, the target moiety is a nucleic acid which is constructed using a specialized primer conjugate. An exemplary primer conjugate is shown in FIG. 3 (top) and comprises a pairing oligonucleotide attached to a crosslinking group for covalent attachment of the pairing oligonucleotide to the capture oligonucleotide. As discussed above, a variety of such crosslinking groups are known in the art and may be used in accord with the invention. In an exemplary embodiment, the crosslinking group is psoralen or a psoralen derivative. The pairing oligonucleotide is covalently linked to a primer via a spacer group. As discussed above, a variety of spacer groups may be used in accord with the invention with the only requirement being that the spacer group is capable of terminating chain extension by the polymerase at the junction of the primer and the spacer group. In an exemplary embodiment, the spacer group is ethylene glycol.

In one embodiment, the psoralen or crosslinking moiety should be adjacent to either the TA or AT combination.

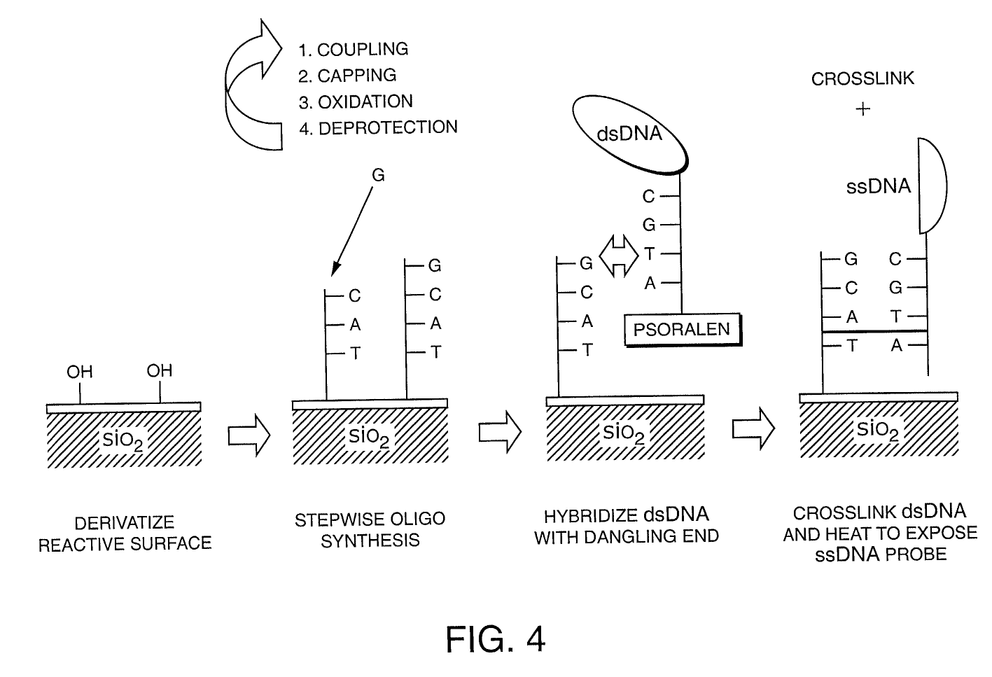

The specialized primer conjugate is then used in combination with a standard PCR primer to amplify a desired region of a nucleic acid. The primer region of the primer conjugate may be designed to comprise any length and sequence necessary to amplify a desired nucleotide sequence by PCR. The resulting conjugate comprises a double stranded nucleic acid, the target moiety, covalently attached to a single stranded oligonucleotide, the pairing oligonucleotide, via the spacer group (see FIG. 3 bottom). The pairing oligonucleotide may then be specifically crosslinked to a capture oligonucleotide with a sequence complementary to the sequence of the pairing oligonucleotide via hybridization between the capture and pairing oligonucleotides. The immobilized double stranded targeting moiety may then be contacted with a test sample to detect an interaction between a molecule in the test sample and the targeting moiety. Alternatively, the double stranded nucleic acid targeting moiety may be denatured to form a single stranded nucleic acid molecule before exposing the targeting moiety to the test sample (see FIG. 4).

VII. Array Construction

The described conjugates may be used in conjunction with the immobilized capture oligonucleotides to produce an array of targeting moieties. The targeting moieties are immobilized onto the array via hybridization between the capture oligonucleotide and the pairing oligonucleotide of the conjugate. In certain embodiments the capture and pairing oligonucleotides are covalent attached using a crosslinking agent. The array preferably comprises a variety of targeting moieties at known, or addressed, locations. The array may then be contacted with a test sample under conditions which promote interaction between molecules in the test sample with one or more of the targeting moieties. The identities of the targeting moieties capable of interacting with a component of the test sample may be determined based on detecting the interaction of the molecules at a particular location on the array.

In one embodiment, the array is constructed using a solid support functionalized with a common capture oligonucleotide. A library of conjugates comprising a common pairing oligonucleotide attached to a variety of targeting moieties is then applied to the functionalized support such that each individual member of the library is localized to a unique position on the array.

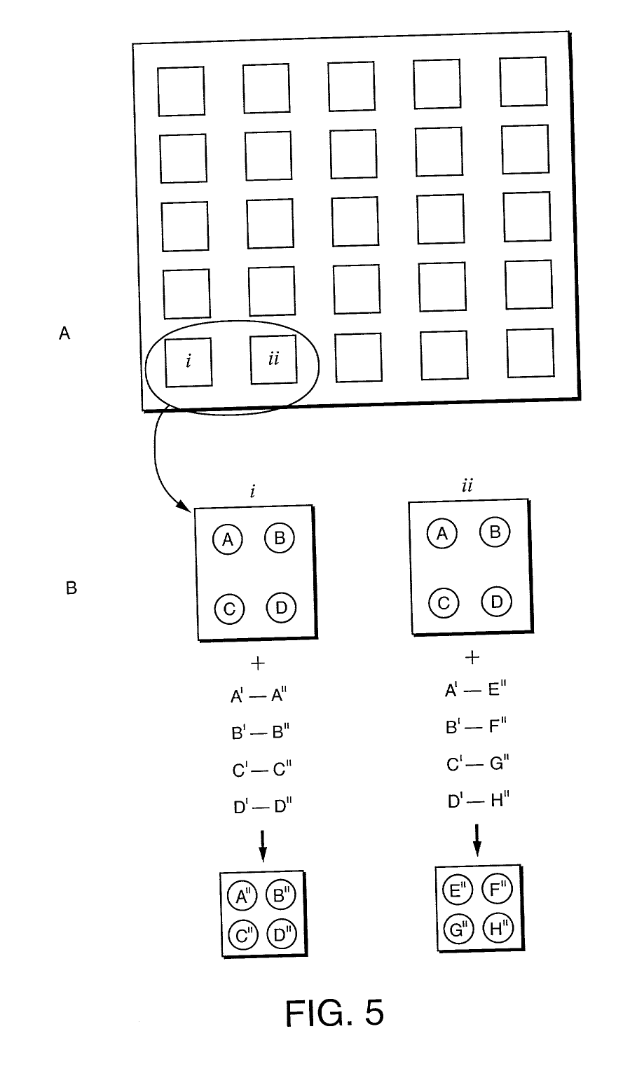

In another embodiment, the array is constructed using pools of conjugates which self assemble to a particular location. In certain embodiments pools may contain from 2 to 1,000, from 2 to 100 or from 2 to 10 different targeting moieties attached to a corresponding number of different pairing oligonucleotides. For example, a solid support may be functionalized with repeating clusters of four capture oligonucleotides with different sequences, called A, B, C and D. A library of conjugates is created wherein 25% of the library is attached to a pairing oligonucleotide complementary to one of the capture oligonucleotides, called A′, B′, C′ and D′, respectively. A mixture of four members of the library each containing a known unique sequence attached to a different pairing oligonucleotide is then applied to one of the clusters of capture oligonucleotides. For example, a mixture of conjugates called A′-A″, B′-B″, C′-C″ and D′-D″ is applied to one of the clusters of A, B, C and D capture oligonucleotides. The A″, B″, C″ and D″ targeting moieties will then self assemble to a known location based on the specific hybridization between A-A′, B-B′, C-C′ and D-D′ capture-pairing oligonucleotide pairs. The next cluster of A, B, C and D capture oligonucleotides is then contacted with another mixture of four members of the library containing four different targeting moieties attached to the same four pairing oligonucleotides. For example, a mixture of conjugates called A′-E″, B′-F″, C′-G″, D′-H″ is applied to the next cluster of A, B, C and D capture oligonucleotides. The E″, F″, G″ and H″ targeting moieties will then self assemble to a known location as before based on the specific hybridization between A-A′, B-B′, C-C′ and D-D′ capture-pairing oligonucleotide pairs (see FIG. 5). This process is repeated until an array of desired complexity has been produced. This pooling method simplifies production of an array by reducing the number of samples which must be applied to the functionalized surface.

In another embodiment, the array is constructed using a library of conjugates which self assemble to a particular location. In this embodiment a solid support is functionalized with a library of capture oligonucleotides localized at defined positions. The functionalized surface is then contacted with a library of conjugates. The conjugate library comprises a library of pairing oligonucleotides, wherein each member is complementary to a different capture oligonucleotide, fused to a library of targeting moieties such that the identity of each targeting moiety fused to each pairing oligonucleotide is known. The entire array will then self assemble to the desired defined locations based on the interactions between the complementary capture and pairing oligonucleotides.

VIII. Exemplary Uses of the Invention

The support bound target moieties of the invention may be used to detect interactions between biomolecules. In particular, one or more targeting moieties may be contacted with a test sample containing one or more test molecules to assay for an interaction between a targeting moiety and one or more test molecules. In various embodiments, the invention may be used to detect nucleic acid-nucleic acid, nucleic acid-polypeptide, polypeptide-polypeptide or polypeptide-small molecule interactions. In other embodiments, the invention may be used in screening assays to identify agonists or antagonists of a particular biomolecular interaction.

Preferably, the test sample comprises a label to permit detection of the interaction. For example, fluorescent labels may be used and many suitable fluorophores are known, including fluorescein, lissamine, phycoerythrin, rhodamine (Perkin Elmer Cetus), Cy2, Cy3, Cy3.5, Cy5, Cy5.5, Cy7, FluorX (Amersham) and others (see, e.g., Kricka, 1992, Academic Press San Diego, Calif.). Additionally, a label other than a fluorescent label may used. For example, a radioactive label, or a pair of radioactive labels with distinct emission spectra, can be used (see Zhao et al., 1995, Gene 156:207; Pietu et al., 1996, Genome Res. 6:492). Descriptions of various methods for using arrays of biomolecules for various applications can be found, for example, in Fodor et al. U.S. Pat. No. 5,871,928.

When the targeting moiety is a nucleic acid, hybridization and wash conditions are chosen so that the population of labeled nucleic acids will specifically hybridize to appropriate, complementary nucleic acids affixed to the solid support via the conjugation oligonucleotide. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches).

Optimal hybridization conditions will depend on the length (e.g., oligomer versus polynucleotide greater than 200 bases) and type (e.g., RNA, DNA, PNA) of labeled nucleic acids and immobilized polynucleotide or oligonucleotide. General parameters for specific (i.e., stringent) hybridization conditions for nucleic acids are described in Sambrook et al., supra, and in Ausubel et al., 1987, Current Protocols in Molecular Biology, Greene Publishing and Wiley-Interscience, New York, which is incorporated in its entirety for all purposes. Non-specific binding of the labeled nucleic acids to the array can be decreased by treating the array with a large quantity of non-specific DNA—a so-called “blocking” step.

When fluorescently labeled probes are used, the fluorescence emissions at each site of a transcript array can be, preferably, detected by scanning confocal laser microscopy. When two fluorophores are used, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, Genome Research 6:639-645). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., 1996, Genome Res. 6:639-645 and in other references cited herein. Alternatively, the fiber-optic bundle described by Ferguson et al., 1996, Nature Biotech. 14:1681-1684, may be used to monitor mRNA abundance levels at a large number of sites simultaneously. Fluorescent microarray scanners are commercially available from Affymetrix, Packard BioChip Technologies, BioRobotics and many other suppliers.

Signals are recorded, quantitated and analyzed using a variety of computer software. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores is preferably calculated. The ratio is independent of the absolute expression level of the cognate gene, but is useful for genes whose expression is significantly modulated by drug administration, gene deletion, or any other tested event.

According to the method of the invention, the relative abundance of an mRNA in two cells or cell lines is scored as a perturbation and its magnitude determined (i.e., the abundance is different in the two sources of mRNA tested), or as not perturbed (i.e., the relative abundance is the same). As used herein, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Present detection methods allow reliable detection of difference of an order of about 2-fold to about 5-fold, but more sensitive methods are expected to be developed.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

In one embodiment of the invention, transcript arrays reflecting the transcriptional state of a cell of interest are made by hybridizing a mixture of two differently labeled sets of cDNAs, to the microarray. One cell is a cell of interest, while the other is used as a standardizing control. The relative hybridization of each cell's cDNA to the microarray then reflects the relative expression of each gene in the two cell. For example, to assess gene expression in a variety of breast cancers, Perou et al. (2000, supra) hybridized fluorescently-labeled cDNA from each tumor to a microarray in conjunction with a standard mix of cDNAs obtained from a set of breast cancer cell lines. In this way, each tumor is compared against the same standard, and may readily be compared against each other.

The present invention also provides the ability to perform diagnostic tests. Diagnostic tests typically are based upon a fingerprint type assay, which tests for the presence of specific diagnostic structural features. Thus, the present invention provides means for viral strain identification, bacterial strain identification, and other diagnostic tests using positionally defined specific reagents. The present invention also allows for determining a spectrum of allergies, diagnosing a biological sample for any or all of the above, and testing for many other conditions.

The present invention provides reagents and methodology for identifying viral strains. The specific reagents may be either antibodies or recognition proteins which bind to specific viral epitopes preferably surface exposed, but may make use of internal epitopes, e.g., in a denatured viral sample. In an alternative embodiment, the viral genome may be probed for specific sequences which are characteristic of particular viral strains. As above, a combination of the two may be performed simultaneously in a single interaction step, or in separate tests, e.g., for both genetic characteristics and epitope characteristics.

Similar techniques will be applicable to identifying a bacterial source. This may be useful in diagnosing bacterial infections, or in classifying sources of particular bacterial species. For example, the bacterial assay may be useful in determining the natural range of survivability of particular strains of bacteria across regions of the country or in different ecological niches.

The present invention provides means for diagnosis of other microbiological and other species, e.g., protozoal species and parasitic species in a biological sample, but also provides the means for assaying a combination of different infections. For example, a biological specimen may be assayed for the presence of any or all of these microbiological species. In human diagnostic uses, typical samples will be blood, sputum, stool, urine, or other samples.

An immobilized set of antigens may be attached to a solid substrate and, instead of the standard skin reaction tests, a blood sample may be assayed on such a substrate to determine the presence of antibodies, e.g., IgE or other type antibodies, which may be diagnostic of an allergic or immunological susceptibility. A standard radioallergosorbent test (RAST) may be used to check a much larger population of antigens.

In addition, an allergy like test may be used to diagnose the immunological history of a particular individual. For example, by testing the circulating antibodies in a blood sample, which reflects the immunological history and memory of an individual, it may be determined what infections may not have been historically presented to the immune system. In this manner, it may be possible to specifically supplement an immune system for a short period of time with IgG fractions made up of specific types of gamma globulins. Thus, hepatitis gamma globulin injections may be better designed for a particular environment to which a person is expected to be exposed. This also provides the ability to identify genetically equivalent individuals who have immunologically different experiences. Thus, a blood sample from an individual who has a particular combination of circulating antibodies will likely be different from the combination of circulating antibodies found in a genetically similar or identical individual. This could allow for the distinction between clones of particular animals, e.g., mice, rats, or other animals.

The present invention provides the ability to fingerprint and identify a genetic individual. This individual may be a bacterial or lower microorganism, as described above in diagnostic tests, or of a plant or animal. An individual may be identified genetically or immunologically, as described.

Genetic fingerprinting has been utilized in comparing different related species in Southern hybridization blots. Genetic fingerprinting has also been used in forensic studies, see, e.g., Morris et al. (1989) J. Forensic Science 34:1311-1317, and references cited therein. As described above, an individual may be identified genetically by a sufficiently large number of probes. The likelihood that another individual would have an identical pattern over a sufficiently large number of probes may be statistically negligible. However, it is often quite important that a large number of probes be used where the statistical probability of matching is desired to be particularly low. In fact, the probes will optimally be selected for having high heterogeneity among the population. In addition, the fingerprint method may make use of the pattern of homologies indicated by a series of more and more stringent washes. Then, each position has both a sequence specificity and a homology measurement, the combination of which greatly increases the number of dimensions and the statistical likelihood of a perfect pattern match with another genetic individual.

As indicated above in the diagnostic tests, it is possible to identify a particular immune system within a genetically homogeneous class of organisms by virtue of their immunological history. For example, a large colony of cloned mice may be distinguishable by virtue of each immunological history. For example, one mouse may have had an immunological response to exposure to antigen A to which her genetically identical sibling may have not been exposed. By virtue of this differential history, the first of the pair will likely have a high antibody titer against the antigen A whereas her genetically identical sibling will have not had a response to that antigen by virtue of never having been exposed to it. For this reason, immune systems may be identified by their immunological memories. Thus, immunological experience may also be a means for identifying a particular individual at a particular moment in her lifetime.

This same immunological screening may be used for other sorts of identifiable biological products. For example, an individual may be identified by her combination of expressed proteins. These proteins may reflect a physiological state of the individual, and would thus be useful in certain circumstances where diagnostic tests may be performed. For example, an individual may be identified, in part, by the presence of particular metabolic products.

In fact, a plant origin may be determined by virtue of having within its genome an unnatural sequence introduced to it by genetic breeders. Thus, a marker nucleic acid sequence may be introduced as a means to determine whether a genetic strain of a plant or animal originated from another particular source.

The present invention provides for the ability to screen for genetic variations of individuals. For example, a number of genetic diseases are linked with specific alleles. See, e.g., Scriber, C. et al. (eds.) (1989) The Metabolic Bases of Inherited Disease, McGraw-Hill, N.Y. In one embodiment, cystic fibrosis has been correlated with a specific gene, see, Gregory et al. (1990) Nature 347:382-386. A number of alleles are correlated with specific genetic deficiencies. See, e.g., McKusick, V. (1990) Genetic Inheritance in Man: Catalogs of Autosomal Dominant, Autosomal Recessive, and X-linked Phenotypes, Johns Hopkins University Press, Baltimore; Ott, J. (1985) Analysis of Human Genetic Linkage, Johns Hopkins University Press, Baltimore; Track, R. et al. (1989) Banbury Report 32: DNA Technology and Forensic Science, Cold Spring Harbor Press, N.Y.; each of which is hereby incorporated herein by reference.

Exemplification

The invention now being generally described, it will be more readily understood by reference to the following examples which are included merely for purposes of illustration of certain aspects and embodiments of the present invention, and are not intended to limit the invention.

The invention generates covalently anchored strands of DNA to a surface by relying on base pairing hybridization as a method for attaching biomolecules to a surface. The immobilization is made permanent by the use of a photoactive crosslinker (if desired). This latter option allows the attachment of double-stranded DNA (dsDNA) to the surface and its subsequent dehybridization to produce end-immobilized single-stranded DNA brushes on the surface (with lengths as great as 400 bp or more) that are available as sites for hybridization and for use in determining the sequence of unknown pieces of DNA. The detailed methods would be compatible with the development of DNA microarray chips by using microspotting techniques.

The process begins by taking a support (often, silicon or glass), derivatizing it with an organosilane reagent to generate a thin organic coating on the substrate that provides chemical sites for further reaction and derivatization. From these sites, oligonucleotide strands are grown from the surface in a stepwise fashion with a selectable sequence using protected nucleotide phosphoramidite reagents commonly used in the solid-phase synthesis of oligonucleotides. This process of synthesis on the substrate surface can be readily automated. We primarily used an automated ABI392 oligosynthesizer and standard cyanoethyl phosphoramidite chemistry, with the exception that Ultramild phosphoramidites were used, as the Ultramild phosphoramidites could be deprotected by mild agents that did not damage the oligo surface. The resulting immobilized oligonucleotide sequence is then deprotected of its side group protecting agents, but done so that the synthesized single-stranded oligonucleotide remains attached to the surface. (Typical sequence lengths on the substrate surface are 10-12 nucleotides (nt), but could be increased to 30-40 nt or so if desired.) The surface density of oligonucleotide chains on the substrate could be tailored by selection of the site density used for growing the oligonucleotides from the surface. This first system forms the base surface for immobilizing biomolecules to the substrate.

Single-stranded pieces of DNA with a complementary structure to that on the surface could be readily adsorbed onto these modified supports with high selectivity. Further, this method worked from dilute aqueous solutions. After hybridization, the adsorbed material was stably located on the surface at room temperature, but could be removed by heating the sample in solution to allow dehybridization of the double-stranded system. This process recovered the original single-stranded material, which could be reused for further hybridization experiments/immobilization.

The pieces of DNA that hybridize to the synthesized ss-DNA brushes on the surface can be covalently immobilized onto this structure through the use of a photoactive crosslinking agent. (We have used psoralen for this purpose.) The cross-linking agent can either be supplied separately from solution as a free species or as an agent that is covalently attached to either the surface or adsorbing stranded before hybridization. After hybridization, irradiation with light covalently links the chains to stably connect it to the substrate.

The combination of hybridization to the surface brush structure and the ability to covalently link the strands together by a photo-cross-linking process availed the formation of ds- and ss-DNA brushes from the surface that were end-immobilized to the substrate. Of particular note was the use of a specialized 5′ PCR primer with a complementary sequence to the initially synthesized ss-DNA brush used for adhesion. The PCR primer was constructed to contain a dangling 5′ end oligo (often a 10-mer) for complementary hybridization, a psoralen terminus for cross-linking, an oligo(ethylene glycol) chain to limit the extent of PCR to the primer, and a traditional primer region for PCR operation. The overall primer was constructed so that PCR produced a piece of ds-DNA (as one example, a chain with roughly 400 bp) that terminated at one end with an overhang comprising the oligo(ethylene glycol) units and a short ss-oligonucleotide sequence used for adhering the ds-DNA to the synthesized surface by hybridization. This strand could be adsorbed onto the substrate through complementary base pairing between the overhang and the substrate surface, and subsequently cross-linked with light to covalently immobilize the ds-DNA strand to the surface as a brush. Heating this sample in solution resulted in the dehybridization of the ds-DNA strand but not in the region formed from the overhang and surface oligonucleotide. The result was a covalently immobilized brush of ss-DNA with sequence lengths that could be readily selected and have a wide range of values (50-400 nt have been achieved). This process for immobilizing the chain has the notable feature that it uses the ds-DNA product from PCR directly and performed the separation into the ss-DNA on the surface directly. In contrast, other methods require that this separation be performed externally prior to immobilization. In our system, the resulting ss-DNA brush offers use for hybridization, reuse by a dehybridization process, and potential use for screening DNA mutations by various methods.

The detailed technology is viewed as being completely compatible with the preparation of DNA microarray chips, and offering advantages in both their preparation (removing the need for a presently required separation step of PCR-ed dsDNA ->ss-DNA) and in their performance (as long strands of ss-DNA in a brush structure are formed on surface with a structure that cannot be produced by other reported methods for such long strands of ss-DNA (e.g., >30 nt).

The above-mentioned immobilization technique covalently attaches dsDNA or ssDNA sequences to silica surfaces via their 5′ terminus. Careful adjustment of the surface oligo density, dsDNA solution concentration and UV photolysis conditions are to control the dsDNA surface surface density. Furthermore, the underlying oligo surface prevents nonspecific adsorption of the dsDNA or ssDNA chain. This system has many advantages over existing methods. The in situ oligo synthesis combined with hybridization to dangling-end dsDNA is a straightforward approach that produces dsDNA and ssDNA surface arrays with controlled surface densities for a wide range of possible applicatons. It does not require chemical crosslinking or enzymatic reactions, which reduce the system consistency and reproducibility and complicate the manufacturing process. Using dsDNA PCR product decreases the production time and high costs associated with the computer-aided design and microlithography techniques required to produce oligo microarrays.

The ssDNA chains are end-attached onto a thin oligo monolayer to form a high-density two-dimensional (2D) array with a brush-like structure. Compared to the horizontally adsorbed cDNA microarrays, this particular ssDNA orientation and terminal attachment method will increase hybridization efficiency, yield, and kinetics, and will minimize mismatches due to the availability of the complete ssDNA sequence for hybridization. In addition, the high specificity of DNA hybridization can be exploited for producing site-specific, spatially-addressable dsDNA or ssDNA combinatorial microarrays through self-assembly of the dangling-end dsDNA chains from a solution mixture. Also, the 2D thin-film structure and absence of polymer gels optimize heat and mass transfer at the surface. Finally, the covalent nature of DNA immobilizing allows for the recycling of the surface array using thermal cycling.

Possible applications for this technology include high-throughput ssDNA microarrays for functional genomics (gene expression assays using cDNA probes), combinatorial DNA libraries for studying DNA-protein, DNA-small molecule and DNA-antisense DNA interactions (for drug discovery), and real-time, portable, solid-state immunological screens for bacteria or viruses. dsDNA arrays can be used for detecting single nucleotide polymorphisms (SNP) by measuring the Tₘ shift in a dsDNA melt curve, using fluorescent intercalating dyes. In addition, the synthesized oligo surfaces can also be utilized as spatially addressable immobilization sites for a variety of biomolecules such as antibodies, small molecules, aptamers, polypeptides, and proteins, resulting in a flexible, multifunctional, solid-state bioassay system.

### EXAMPLE 1

### OH—Silane Solid Support Surfaces

The SiO₂ surfaces were prepared by cutting 3 cm×1 cm chips from <100> silicon wafers. Immerse silicon surfaces for 2 hours in washing solution composed of 40 grams of sodium hydroxide in 150 ml of water and 200 ml of 95% ethanol, rinse with MilliQ water followed by 95% ethanol, and dry with nitrogen gas stream. Immerse cleaned silicon surfaces in 0.1 wt % 3-hydroxypropyl 4-trimethoxysilyl butyramide in 95% ethanol solution for 8-16 hours, rinse with 95% ethanol, dry with nitrogen gas stream. Rinse with 95% ethanol, and dry with nitrogen gas stream. This route can be used to obtain surface hydroxy group densities that result in oligo surface densities of 5×10⁻¹¹ moles/cm².

### EXAMPLE 2

### Trichlorosilane Solid Support Surfaces

The SiO₂ surfaces were prepared by cutting 3 cm×1 cm chips from <100> silicon wafers. Immerse silicon surfaces for 2 hours in washing solution composed of 40 grams of sodium hydroxide in 150 ml of water and 200 ml of 95% ethanol, rinse with MilliQ water followed by 95% ethanol, and dry with nitrogen gas stream. Immerse clean silicon surfaces in mixtures of octyltrichlorosilane or dodecyltrichlorosilane, and trichloroacetylundecyltrichlorosilane in dry xylene (total concentration of trichlorosilanes=20 μl in 20 ml of solvent, or about 0.1 vol %) for 30 minutes to 1 hour, rinse with acetone followed by 95% ethanol, and dry with nitrogen gas stream. Deprotect with hydroxy groups by removing the trichloroacetyl groups from the silanized silicon surfaces, by immersion in a solution of 0.05 M potassium carbonate dissolved in a 1:1 ratio of water to methanol, for 15 minutes. An alternative deprotection protocol is to heat to 90° C. in pH=3-4 water for 2 hours.

### EXAMPLE 3

### Synthesis of Surface Immobilized Oligonucleotides

Standard phosphoramidite synthesis chemistry is employed with the derivatized silicon surfaces as the substrate/support: Insert hydroxyl terminated silane derivatized silicon chips into empty synthesis columns. The coupling reaction time is over 75 seconds to ensure optimal coupling efficiency, and remove the 5′ DMT protecting group to expose a 5′ hydroxy group. For the bases adenine (A), guanine(G), and cytosine(C), use Ultramild® phosphoramidites for mild deprotection. Wash the chips in the column with acetonitrile for 60 seconds, then dry with nitrogen gas for 60 seconds. Wash chips with acetone followed by 95% ethanol, and dry with nitrogen stream. Immerse the chips in 0.05M potassium carbonate in dry methanol for 2 hours, followed by 95% ethanol rinse and dried with nitrogen stream to remove the base protecting groups,. To remove the cyanoethyl groups immerse chips in 20 v/v % piperidine in dimethyl formamide for 2 hours, followed by 95% ethanol rinse and dried with nitrogen stream. Coupling efficiency increases with decreasing density of hydroxy reactive sites on the derivatized silicon surfaces, with 99+% efficiency when density is lower than 5×10⁻¹¹ moles/cm².

### EXAMPLE 4

### Synthesis of Surface Immobilized Oligonucleotides with Psoralen Attachment

Standard phosphoramidite synthesis chemistry is employed with the derivatized silicon surfaces as the substrate/support: Insert hydroxyl terminated silane derivatized silicon chips into empty synthesis columns. Add on a C2 psoralen phosphoramidite, with coupling time adjusted to 900 seconds to maximize coupling efficiency and remove the 5′ DMT protecting group to expose a 5′ hydroxy group. For the bases adenine (A), guanine(G), and cytosine(C), use Ultramild® phosphoramidites for mild deprotection. Wash the chips in the column with acetonitrile for 60 seconds, then dry with nitrogen gas for 60 seconds. Wash chips with acetone followed by 95% ethanol, and dry with nitrogen stream. Immerse the chips in 0.05M potassium carbonate in dry methanol for 2 hours, followed by 95% ethanol rinse and dried with nitrogen stream to remove the base protecting groups,. To remove the cyanoethyl groups immerse chips in 20 v/v % piperidine in dimethyl formamide for 2 hours, followed by 95% ethanol rinse and dried with nitrogen stream. Coupling efficiency increases with decreasing density of hydroxy reactive sites on the derivatized silicon surfaces, with 99+% efficiency when density is lower than 5×10⁻¹¹ moles/cm².

### EXAMPLE 5

### Determination of Oligo Surface Density by XPS

Iodinated Uracil phosphoramidites (Glen Research) are used to label to characterize oligo density. The iodine signal at 633 eV and 620 eV. The iodine peak area is quantified and compared to the peak area of samples to a standard. The XPS iodine standard is a 1000 Angstrom gold surface with a <111> crystal orientation evaporated onto silicon. The gold surface is immersed for 24 hours in a 0.1 M potassium iodide aqueous solution so that a iodide adlayer is formed on the gold substrate with coverage=0.3 and density=9×10⁻¹⁰ moles/cm².

### EXAMPLE 6

### Synthesis of the 5′ Psoralen Linked Dangling-End dsDNA

Use a custom-synthesized oligo with 5′ C2 psoralen entity, followed by a 10 mer sequence (for hybridization to the surface-immobilized complementary oligo chain), followed by a triethylene oxide spacer, followed by a 20 mer that acts as a 5′ primer during the polymerase chain reaction (PCR). The ethylene oxide spacer is used to prevent the Taq polymerase enzyme from filling in the 10 mer sequence during the PCR reaction, to create a single strand 10 mer dangling end that can hybridize to the surface oligo chains. Run a PCR reaction with the custom 5′ primer and the appropriate dsDNA template, 3′ primer, mixture of nucleotide triphosphates (2 mM of A, T, G and 1.5 mM of C) and Taq polymerase. Use a spin column to purify the dangling end dsDNA PCR product, and lyophilize the dsDNA with a speed vac or use ethanol precipitation to purify the dsDNA. Resuspend the solid dsDNA in 7×sodium saline citrate (SSC) buffer (1:2 volume ratio of 20×SSC to filtered double deionized water) containing 0.1 wt % sodium dodecyl sulfate (SDS). The concentration should be at least 200 μg/ml and preferably more than 500 μg/ml to optimize hybridization.

### EXAMPLE 7

### Hybridization to the Surface Immobilized Oligo Chains

For hybridization to complementary oligo sequences immerse oligo chips in a solution of 0.5 μM oligo concentration in 7×SSC (sodium saline citrate) buffer with 0.1 wt % SDS at 4° C. for 24 hours, followed by a rinse with a 4° C. Tris-ethylenediaminetetraacetic acid (TE) buffer, and dry with a nitrogen stream. For hybridization to dsDNA chains immerse oligo chips in a solution of 500 μg/ml dsDNA concentration in 7×SSC buffer with 0.1 wt % SDS at 4° C. for 48 hours, followed by a rinse with a 4° C. TE buffer, and dry with a nitrogen stream. To optimize hybridization the sequence of the surface-immobilized oligo can be manipulated. Longer chain lengths, up to 20 mer, and increased GC content (with consequent increase in dsDNA melting temperature) will both improve hybridization yields. For hybridization to solution-phase oligos increased solution oligo concentration will improve hybridization rate and yield. For hybridization of dangling-end dsDNA, increasing the solution dsDNA concentration, and the addition of 10 wt % dextran sulfate, both help increase the hybridization yield of dsDNA by up to 125%. The density of reactive hydroxy groups on the derivatized silicon surfaces affects the hybridization yields, and the optimal density of the oligos is between 3-4×10⁻¹¹ moles/cm².

### EXAMPLE 8

### Psoralen Crosslinking

Immerse hybridized chips in 7×SSC solution on ice, and illuminate the chips with a 2×15 Watt low pressure Hg lamp as the UV light source. Use a 2 hour illumination time. The hybridized chips are immersed in TE buffer with 0.2 wt % SDS and heated to 60° C. for 1 hour to remove the uncrosslinked dsDNA or oligo. The immobilized dsDNA is then immersed in TE buffer with 0.2 wt % SDS and heated to 90° C. for 1 hour to remove one of the ssDNA strands and expose the probe ssDNA attached to the silicon surface at its 5′ end. After SDS wash, rinse the samples with MQ water and dry with a nitrogen gas stream. About 30% of hybridized dsDNA is crosslinked to surface and remains after heating. It becomes ssDNA if heated to 90° C. or remains dsDNA if heated to 60° C., and the final form depends on the desired application. There are a variety of potential applications for both ssDNA and dsDNA immobilized onto a surface in a spatially addressable manner. The dsDNA surfaces could be used to study DNA interactions with proteins, small molecules and other biomolecules, while heating the dsDNA surfaces up to observe the melting profile may possibly be used to discern SNP within the dsDNA sequence for polymorphism screening and research. The ssDNA attached to the surface could be used as a DNA probe library for rapid forensics and pathogen screens as well as quantitative gene expression studies.

### EXAMPLE 9

### Self Assembling Array

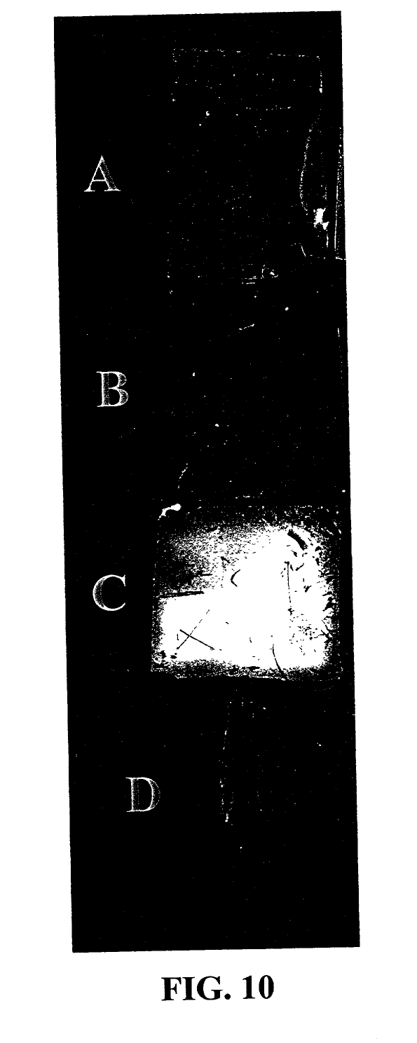

Three different capture oligonucleotides were used to functionalize a solid support. The capture oligonucleotides had the sequences A, B or C wherein A is a polyT sequence and B and C had sequences complementary to a pairing oligonucleotide of sequence A′ and B′. A mixture of conjugates was then applied to the surface of the array covering each of the regions functionalized with A, B and C capture oligonucleotides. The conjugate mixture was also applied to a surface which was not functionalized with an oligonucleotide (D). The conjugate mixture contained two conjugates. The B′ conjugate comprised a dsDNA sequence labeled with Cy3 fused to a pairing oligonucleotide which was complementary to the B capture oligonucleotide. The C′ conjugate comprised a dsDNA sequence labeled with Cy5 fused to a pairing oligonucleotide which was complementary to the C capture oligonucleotide. The array was incubated with the conjugate mixture to allow hybridization to occur and then washed to remove any unbound conjugates. Cy3 and Cy5 fluorescence images were used to assay binding of the conjugates to the array. The Cy5 image is shown in FIG. 10 and demonstrates that C′ conjugate was localized specifically to the complementary C capture oligonucleotide. Similarly, the Cy3 image showed specific localization of the B′ conjugate to the complementary B capture oligonucleotide. The signal to noise ratio for specific localization of the conjugates ranged from 10:1 to 37:1. The fluorescence intensity due to nonspecific adsorption of the conjugate to the array was <1%. Similarly, the fluorescence intensity due to mismatch or adsorption of the conjugate to the array was <5%.

Additional information may be found in U.S. Pat. Nos. 5,919,523, 5,642,711, 5,482,867, 5,451,683, 5,412,087, and 5,252,743.

All of the references cited above are hereby incorporated by reference herein.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A support-bound probe, comprising
a capture oligonucleotide sequence covalently attached to the surface of a solid support, and
a conjugate, comprising
a target moiety,
a pairing oligonucleotide sequence which specifically hybridizes to the surface-bound oligonucleotide sequence, and which is covalently attached to the target moiety, and
a crosslinking moiety covalently bound to the pairing oligonucleotide sequence and to either the surface-bound oligonucleotide sequence or the surface of the solid support.

2. The support-bound probe of claim 1, wherein the target moiety is covalently attached to the pairing oligonucleotide sequence through a linking moiety.

3. The support-bound probe of claim 2, wherein the linking moiety is ethylene glycol.

4. The support-bound probe of claim 1, wherein the target moiety is directly attached to the pairing oligonucleotide sequence to form a continuous oligonucleotide sequence.

5. The support-bound probe of claim 1, wherein the crosslinking moiety is psoralen.

6. The support-bound probe of claim 1, wherein the target moiety is a single-stranded or double-stranded nucleotide sequence.

7. The support-bound probe of claim 1, wherein the target moiety is a protein, peptide, or oligopeptide.

8. The support-bound probe of claim 1, wherein the target moiety is a small molecule.

9. The support-bound probe of claim 1, wherein the pairing oligonucleotide sequence comprises between 3 and 50 nucleotides.

10. The support-bound probe of claim 1, wherein the pairing oligonucleotide sequence comprises between 3 and 20 nucleotides.

11. The support-bound probe of claim 1, wherein the pairing oligonucleotide sequence comprises between 3 and 10 nucleotides.

12. A solid support bearing an array of support-bound probes of claim 1 on the surface thereof.

13. The solid support of claim 12, bearing at least 50 probes on the surface thereof.

14. The solid support of claim 12, bearing at least 100 probes on the surface thereof.

15. The solid support of claim 12, bearing at least 500 probes on the surface thereof.

16. The solid support of claim 12, bearing a probe density of between 1-12×10⁻¹¹ moles per cm².

17. The solid support of claim 12, bearing a probe density between 2-6×10⁻¹¹ moles per cm².

18. The solid support of claim 12, bearing a probe density between 3-4×10⁻¹¹ moles per cm².

19. The solid support of claim 12, wherein the array comprises a library of target moieties.

20. A probe comprising
a target moiety,
an oligonucleotide sequence covalently attached to the target moiety, and
a crosslinking moiety covalently bound to the oligonucleotide sequence and capable of forming a covalent bond to a surface or to an oligonucleotide sequence under predetermined conditions.

21. The probe of claim 20, wherein the predetermined conditions are exposure to light.

22. A solid support having an array of surface-bound capture oligonucleotides, wherein each oligonucleotide is covalently bound to a crosslinking moiety capable of forming a covalent bond to an oligonucleotide sequence complementary to the oligonucleotide.

23. The solid support of claim 22, wherein the surface-bound capture oligonucleotides comprise between 3 and 50 nucleotides.

24. The solid support of claim 22, wherein the support is functionalized with multiple surface-bound capture oligonucleotides all having the same sequence.

25. The solid support of claim 22, wherein the support is functionalized with multiple surface-bound capture oligonucleotides having a variety of sequences localized to a defined location on the solid support.

26. The solid support of claim 22, wherein the support is functionalized with repeating units of clusters of surface-bound capture oligonucleotides having a variety of sequences localized to a variety of defined location on the solid support.

27. A method for linking a probe to a solid support comprising
providing a solid support having an array of surface-bound oligonucleotides,
hybridizing to a surface-bound oligonucleotide a probe comprising a pairing oligonucleotide sequence complementary to the surface-bound oligonucleotide sequence and a target moiety, and
forming a covalent bond between the pairing oligonucleotide sequence and either the surface-bound oligonucleotide or the solid support.

28. The method of claim 27, wherein forming a covalent bond includes forming a covalent bond between a crosslinking moiety on the pairing oligonucleotide sequence and the surface-bound oligonucleotide sequence.

29. The method of claim 27, wherein forming a covalent bond includes forming a covalent bond between a crosslinking moiety on the pairing oligonucleotide sequence and the surface.

30. The method of claim 27, wherein forming a covalent bond includes forming a covalent bond between a crosslinking moiety on the surface-bound oligonucleotide sequence and the pairing oligonucleotide sequence.

31. A conjugate primer comprising
a PCR primer,
an oligonucleotide sequence covalently attached to the PCR primer, and a crosslinking moiety covalently bound to the oligonucleotide sequence and capable of forming a covalent bond to a surface or to an oligonucleotide sequence under predetermined conditions.

32. The conjugate primer of claim 31, wherein the PCR primer is covalently attached to the oligonucleotide sequence through a linking moiety.

33. The conjugate primer of claim 32, wherein the linking moiety is ethylene glycol.

34. The conjugate primer of claim 31, wherein the crosslinking moiety is psoralen.

35. A method for forming a self-assembling array of a library of target moieties comprising
providing a solid support having an array of surface-bound capture oligonucleotides wherein each capture oligonucleotide having a unique sequence is localized at one or more defined positions on the solid support,
contacting the array of surface-bound capture oligonucleotides with a mixture of conjugates comprising a library of target moieties fused to pairing oligonucleotides with sequences complementary to the surface-bound capture oligonucleotides;
forming a covalent bond between the pairing oligonucleotide sequence and either the surface-bound capture oligonucleotide or the solid support.

36. The method of claim 35, wherein the solid support is functionalized with a library of capture oligonucleotides and is contacted with a complementary library of pairing oligonucleotides fused to a library of target moieties.

37. The method of claim 35, wherein the solid support is functionalized with repeating clusters of capture oligonucleotides with unique sequences,
wherein each cluster is contacted with a set of conjugates comprising pairing oligonucleotides capable of specifically hybridizing to each unique capture oligonucleotide sequence in the cluster, and
wherein each cluster is contacted with a different set of conjugates comprising different targeting moieties fused to a common set of pairing oligonucleotides.

38. The method of claim 35, wherein the cluster comprises from 2 to 1000 different capture oligonucleotides.

39. The method of claim 35, wherein the cluster comprises from 2 to 100 different capture oligonucleotides.

40. The method of claim 35, wherein the cluster comprises from 2 to 50 different capture oligonucleotides.

41. The method of claim 35, wherein the cluster comprises from 2 to 10 different capture oligonucleotides.

42. A method for producing a double stranded DNA sequence with a single stranded overhang comprising
contacting a target DNA sequence with a pair of oligonucleotide primers wherein one of the primers is covalently attached to a single stranded oligonucleotide through a linking moiety, and
amplifying the target DNA using PCR.

43. The method of claim 42, wherein the linking moiety is ethylene glycol.

44. The method of claim 42, wherein the single stranded oligonucleotide further comprises a covalently attached crosslinking moiety.

45. The method of claim 44, wherein the crosslinking moiety is psoralen.

46. The method of claim 42, wherein the single stranded oligonucleotide is attached to the PCR primer which is extended to produce the sense strand of the target DNA.

47. The method of claim 42, wherein the 3′ end of the single stranded oligonucleotide is attached to the 5′ end of the PCR primer which is extended to produce the sense strand of the target DNA.
